(19) United States
(12) Patent Application Publication
(10) Pub. No.: US 2026/0273168 A1
(43) Pub. Date: Sep. 17, 2026

(54) SYSTEMS AND METHODS FOR MEASURING GEOMETRIC DIMENSIONS OF A SYRINGE AND/OR A PLUNGER STOPPER

(71) Applicant: AMGEN INC., Thousand Oaks, CA (US)

(72) Inventors: Andrew Gu, Thousand Oaks, CA (US); Moris Saravia, Thousand Oaks, CA (US); Rathi Harish, Thousand Oaks, CA (US); Abby Roulier, Thousand Oaks, CA (US); Silvio Santinelli, Thousand Oaks, CA (US); Shree Thoka, Thousand Oaks, CA (US)

(21) Appl. No.: 19/166,916

(22) PCT Filed: Mar. 14, 2024
(86) PCT No.: PCT/US2024/019862

§ 371 Date: Sep. 19, 2025

Related U.S. Application Data
(60) Provisional application No. 63/453,340, filed on Mar. 20, 2023.

(51) Int. Cl. A61M 5/00; G06T 7/60

(52) U.S. Cl. CPC A61M 5/008; G06T 7/60

(57) ABSTRACT

Systems and methods are provided for generating geometric dimensions of a syringe or a plunger stopper. The geometric dimensions may be based on an at least one image of at least a portion of the syringe or plunger stopper. Fixtures are provided for positioning the syringe or plunger stopper proximate an associated imaging device.

### CROSS-REFERENCE TO RELATED APPLICATIONS

Priority is claimed to U.S. Provisional Patent Application No. 63/453,340, filed Mar. 20, 2023, the entire contents of which are hereby incorporated by reference herein.

### FIELD OF DISCLOSURE

The present disclosure relates generally to measuring geometric dimensions of a syringe and/or a plunger stopper and, more specifically, to measuring geometric dimensions of a syringe and/or a plunger stopper based on associated digital images.

### BACKGROUND

Numerous drug products are manufactured and stored in syringes. Associated prefilled syringes may be manufactured to high quality standards. Prefilled syringes often include a plunger stopper that fits tightly within a barrel of the syringe. Some geometric dimensions of both the syringe (e.g., a barrel inner diameter, a luer outer diameter, a dose line location, a flange diameter, a barrel length, a flange thickness, a barrel outer diameter, etc.) and the plunger stopper (e.g., an overall length, a width, an angular alignment, etc.) are tightly controlled.

The geometric dimensions of the syringe and the geometric dimensions of the plunger stopper are typically measured manually using, for example, a ruler, calibers, etc. Manually measuring geometric dimensions of a syringe and a plunger stopper are time consuming and subject to human error. Associated records of the geometric dimensions are also subject to error.

### SUMMARY

Embodiments described herein relate to systems and methods for measuring geometric dimensions of a syringe and/or a plunger stopper.

As described herein, a syringe fixture can be configured to align a central syringe axis of a syringe with respect to a central image axis of an imaging system. The syringe fixture includes a support block defining a V-shaped notch, a first planar support surface, and a second planar support surface. The first planar support surface having a first edge. The second planar support surface having a second edge, wherein at least a portion of the second edge conjoins at least a portion of the first edge, and wherein the second planar support surface is perpendicular to the first planar support sur-face. The syringe fixture also includes a syringe securing mechanism coupled to the support body and configured to secure a syringe in a fixed position in the V-shaped notch such that the syringe occupies a first orientation when the support block is positioned on the first planar support surface and a second orientation perpendicular to the first orientation when the support block is positioned on the second planar support surface.

A system for measuring at least one geometric dimension of a syringe includes a syringe imaging system having a central imaging axis and a stage planar surface. The system also includes a syringe fixture configured to secure a syringe in a first orientation relative to the central imaging axis when the support block is positioned on the first planar support surface. The system further includes an image sensor configured to capture a first image of at least a portion of the syringe when secured by the syringe fixture and positioned on the first planar surface. The system yet further includes one or more processors configured to generate at least one geometric measurement of the syringe based upon at least the first image.

A method for measuring a geometric dimension of a syringe includes securing a syringe in a syringe fixture. The method also includes placing the syringe and syringe fixture in a first position on a stage planar surface of an imaging system, the syringe having a central syringe axis occupying a first predetermined orientation relative to a central image axis of the imaging system. The method further includes acquiring, with an image sensor of the imaging system, at least one image of at least a portion of the syringe. The method yet further includes calculating, with a processor of the imaging system, at least a first geometric dimension of the syringe based on the at least one image.

A plunger stopper fixture can be configured to align a central plunger stopper axis of a plunger stopper with respect to a central image axis of an imaging system. The plunger stopper fixture includes a base comprising an imaging system attachment configured to be secured in a fixed position to a stage of an imaging system having a central image axis. The plunger stopper fixture includes a plunger stopper rotation mechanism coupled to the base, the plunger stopper rotation mechanism comprising a dial rotatable about a dial axis and a rod extending from the dial along the dial axis. The rod having a distal end disposed away from the dial. The distal end of the rod having a plunger stopper retaining tip configured to secure a plunger stopper to the plunger stopper fixture and in alignment with the central image axis. The plunger stopper rotation mechanism configured such that a central plunger stopper axis of the plunger stopper is coaxially aligned with the dial axis and rotation of the dial about the dial axis rotates the plunger stopper about the central plunger stopper axis.

A system for measuring at least one geometric dimension of a plunger stopper includes a plunger stopper imaging device having a central imaging axis. The system also includes a plunger stopper fixture having a plunger stopper rotation mechanism, wherein the plunger stopper fixture is configured to secure the plunger stopper relative to the plunger stopper imaging device with a central plunger stopper axis aligned with the central image axis, wherein the plunger stopper rotation mechanism is configured to rotate the plunger stopper around the central plunger stopper axis, wherein the plunger stopper imaging device captures a first image of at least a portion of the plunger stopper with the plunger stopper oriented at first rotational angle around the central plunger stopper axis, and wherein the plunger stopper imaging device captures a second image of at least a portion of the plunger stopper with the plunger stopper oriented at second rotational angle around the central plunger stopper axis. The system further includes a geometric measurement generation device configured to generate at least one geometric measurement of the plunger stopper based on the first image and the second image.

A method for measuring at least one geometric dimension of a plunger stopper includes aligning a central plunger stopper axis of a plunger stopper at a fixed angle with respect to a central image axis of an imaging device, and at an initial rotational angle around the central plunger stopper axis. The method also includes capturing, with an imager, a first image of a profile view of the plunger stopper with the plunger stopper oriented at the initial rotational angle around the central plunger stopper axis. The method further includes determining, with a processor, a first value for the at least one geometric dimension of the plunger stopper based on the first image. The method yet further includes rotating the plunger stopper around the central plunger stopper axis to a second rotational angle. The method also includes capturing, with the imager, a second image of a profile view of the plunger stopper with the plunger stopper oriented at the second rotational angle. The method further includes determining, with the processor, a second value for the at least one geometric dimension of the plunger stopper based on the second image.

Novel systems and methods are provided for measuring geometric dimensions of a syringe and/or a plunger stopper.

### BRIEF DESCRIPTION OF THE DRAWINGS

The skilled artisan will understand that the figures described herein are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIGS. 1A and 1B depict an example syringe measurement system.

FIG. 2 depicts an example user interface display.

FIG. 3 depicts an example syringe fixture with a syringe having a central syringe axis parallel to a central image axis.

FIG. 4 depicts an example syringe fixture with a syringe having a central syringe axis perpendicular to a central image axis.

FIG. 5 depicts an example syringe fixture with a syringe having a central syringe axis parallel to a central image axis.

FIG. 6 depicts an example syringe fixture with a syringe having a central syringe axis parallel to a central image axis for measuring a barrel ID.

FIG. 7 depicts an example user interface display of a syringe fixture with a syringe having a central syringe axis parallel to a central image axis.

FIG. 8 depicts an example syringe fixture with a syringe having a central syringe axis parallel to a central image axis and proximate a light probe.

FIG. 9 depicts an example user interface display of a syringe fixture with a syringe having a central syringe axis parallel to a central image axis.

FIG. 10 depicts an example syringe fixture with a syringe having a central syringe axis perpendicular to a central image axis for luer OD measurement.

FIG. 11 depicts an example user interface display of a syringe fixture with a syringe having a central syringe axis parallel to a central image axis.

FIG. 12 depicts an example user interface display of a syringe fixture with a syringe having a central syringe axis parallel to a central image axis.

FIG. 13 depicts an example user interface display of a syringe fixture with a syringe having a central syringe axis perpendicular to a central image axis for dose line location measurement.

FIG. 14 depicts an example user interface display of a syringe fixture with a syringe having a central syringe axis perpendicular to a central image axis.

FIG. 15 depicts an example syringe fixture with a syringe having a central syringe axis parallel to a central image axis.

FIG. 16 depicts an example syringe fixture with a syringe having a central syringe axis parallel to a central image axis and syringe flange orientation.

FIG. 17 depicts an example user interface display of a syringe fixture with a syringe having a central syringe axis parallel to a central image axis and syringe flange diameter.

FIG. 18 depicts an example user interface display of a syringe fixture with a syringe having a central syringe axis parallel to a central image axis and syringe flange diameter.

FIG. 19 depicts an example syringe fixture with a syringe having a central syringe axis perpendicular to a central image axis for syringe barrel length measurement.

FIG. 20 depicts an example user interface display of a syringe fixture with a syringe having a central syringe axis perpendicular to a central image axis for syringe barrel length measurement.

FIG. 21 depicts an example user interface display of a syringe fixture with a syringe having a central syringe axis perpendicular to a central image axis for syringe barrel length measurement.

FIG. 22 depicts an example syringe fixture with a syringe having a central syringe axis perpendicular to a central image axis for syringe for syringe flange thickness measurement

FIG. 23 depicts an example user interface display of a syringe fixture with a syringe having a central syringe axis perpendicular to a central image axis for syringe flange thickness measurement

FIG. 24 depicts an example user interface display of a syringe fixture with a syringe having a central syringe axis perpendicular to a central image axis for syringe barrel OD measurement.

FIG. 25 depicts an example user interface display of a syringe fixture with a syringe having a central syringe axis perpendicular to a central image axis for syringe barrel OD measurement.

FIGS. 26A-G is an example method of measuring geometric dimensions of a syringe.

FIG. 27 depicts an example plunger stopper measurement system.

FIG. 28 depicts an example plunger stopper measurement system with plunger stopper fixture.

FIG. 29 depicts an example plunger stopper measurement system with a plunger stopper fixture and a plunger stopper having a central plunger stopper axis perpendicular to a central image axis.

FIG. 30 depicts an example user interface display of a plunger stopper fixture with a plunger stopper having a central plunger stopper axis perpendicular to a central image axis.

FIG. 31 depicts a dial indicator of a plunger stopper fixture reoriented from 90° to 70°.

FIG. 32 depicts an example user interface display of a plunger stopper fixture with a plunger stopper having a central plunger stopper axis perpendicular to a central image axis.

FIG. 33 depicts an example user interface display of a plunger stopper fixture with a plunger stopper having a central plunger stopper axis perpendicular to a central image axis.

FIG. 34 depicts an example user interface display of a plunger stopper fixture with a plunger stopper having a central plunger stopper axis perpendicular to a central image axis.

FIG. 35 depicts an example method of measuring geometric dimensions of a plunger stopper.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

Systems and methods are provided for measuring geometric dimensions of a syringe (e.g., a barrel inner diameter, a luer outer diameter, a dose line location, a flange diameter, a barrel length, a flange thickness, a barrel outer diameter, etc.) and a plunger stopper (e.g., an overall length, a width, an angular alignment, etc.). Syringe fixtures are provided for aligning a central syringe axis with a central image axis of an associated imaging device. Geometric dimensions of the syringe are measured based on one or more images of the syringe and/or a light probe measurement. A plunger stopper fixture is provided for aligning a central plunger stopper axis with the central image axis. Geometric dimensions of the plunger stopper are measured based on images of the plunger stopper.

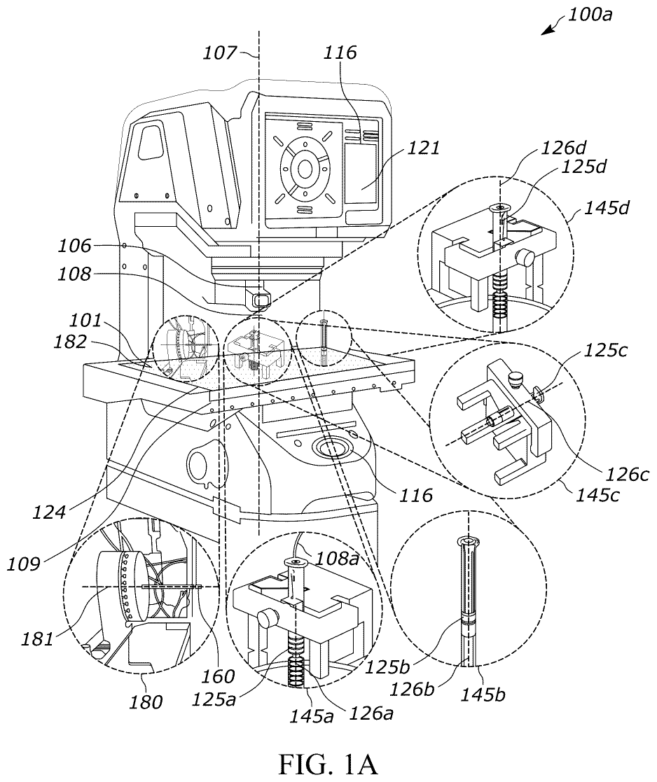

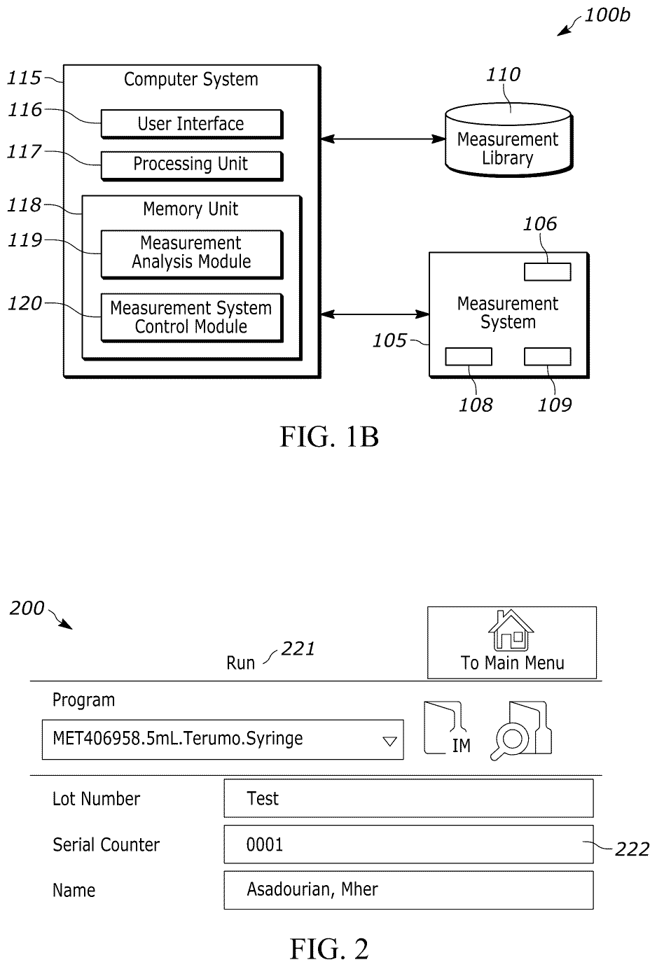

FIGS. 1A and 1B depict an example system 100a,b for measuring geometric dimensions of a syringe and a plunger stopper. System 100a,b includes a measurement system 105 having at least one imaging device 106, at least one light probe 108 (e.g., a light-based proximity sensor, a light emitter, a light receiver, etc.), at least one illumination source 109 (e.g., a backlight, a direct light, an angled light, a darkfield light, a coaxial light, etc.), and at least one user interface 116. The user interface 116 may include a plurality of user interface displays 121 related to measuring geometric dimension of a syringe 125a-d and/or a plunger stopper 160. The imaging device 106 includes a central image axis 107.

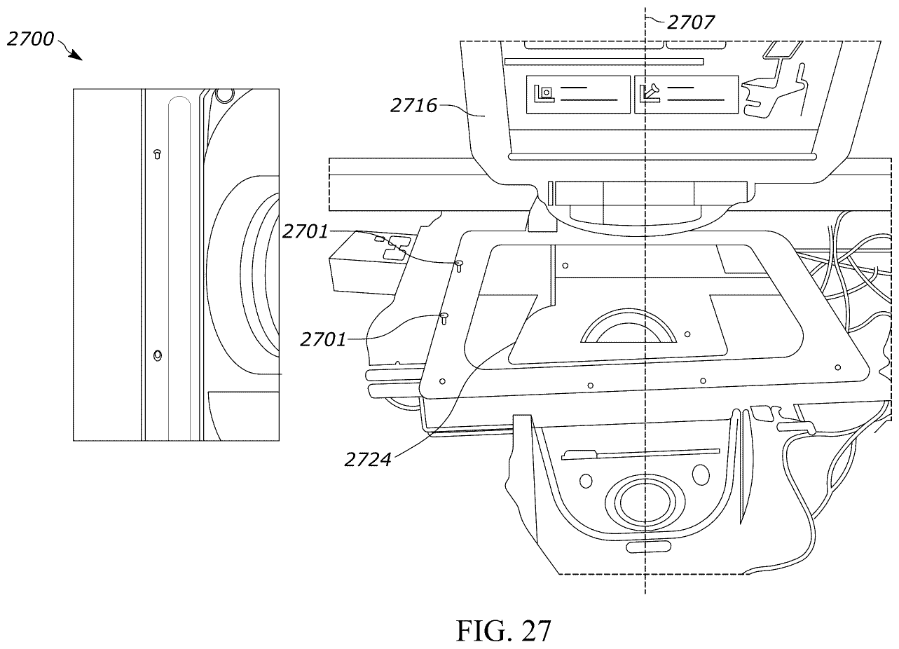

The system 100 also includes a support platform 124, and a plunger stopper fixture attachment 101. The system 100 also includes a plunger stopper fixture 180 having an imaging device attachment 182. When the imaging device attachment 182 of the plunger stopper fixture 180 is attached to the plunger stopper fixture attachment 101, a central plunger stopper axis 181 of the plunger stopper 160 is aligned and possibly intersecting with the central image axis 107. For example, the central plunger stopper axis 181 of the plunger stopper 160 may be aligned perpendicular to the central image axis 107. Further details related to measuring geometric dimensions (e.g., an overall length, a width, an angular alignment, etc.) of the plunger stopper 160 are described with reference to FIGS. 27-35.

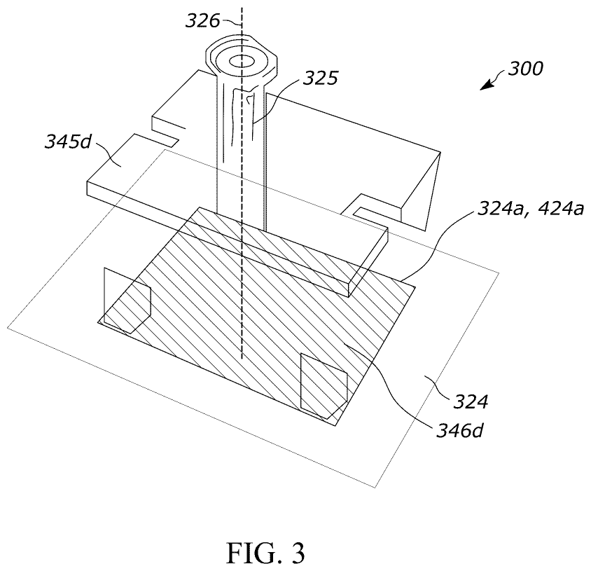

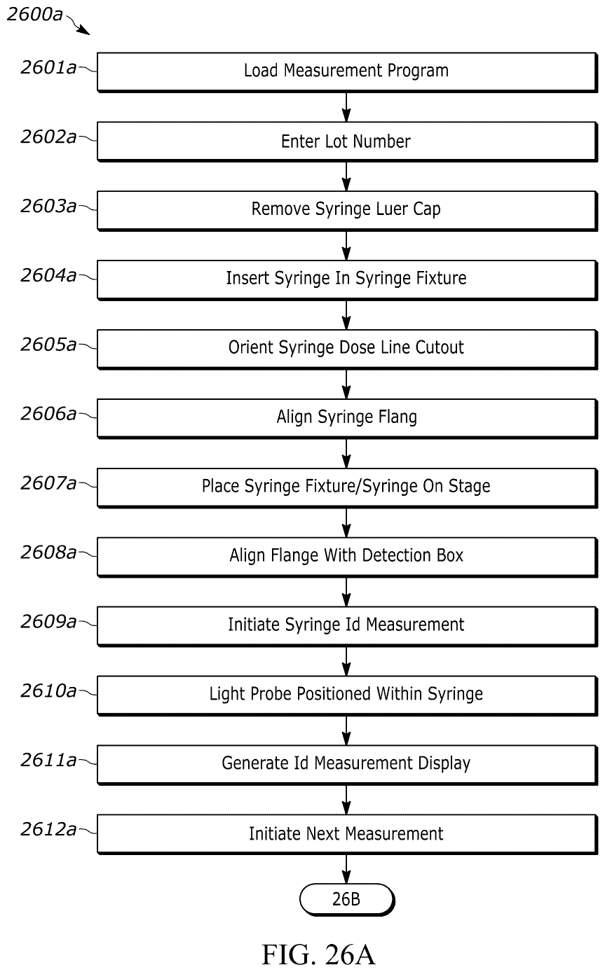

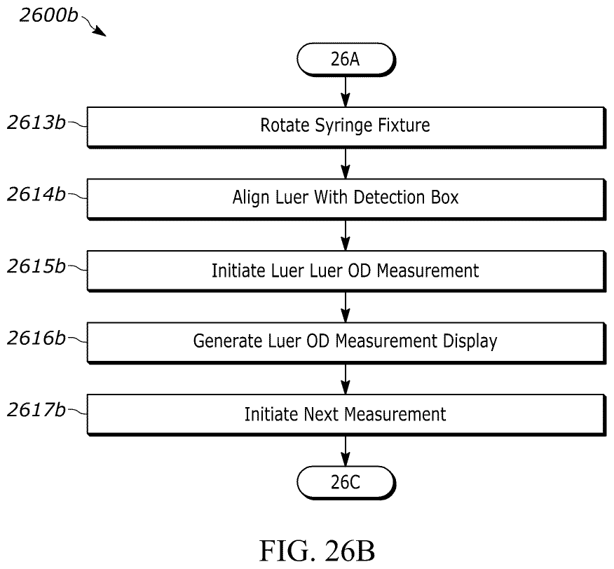

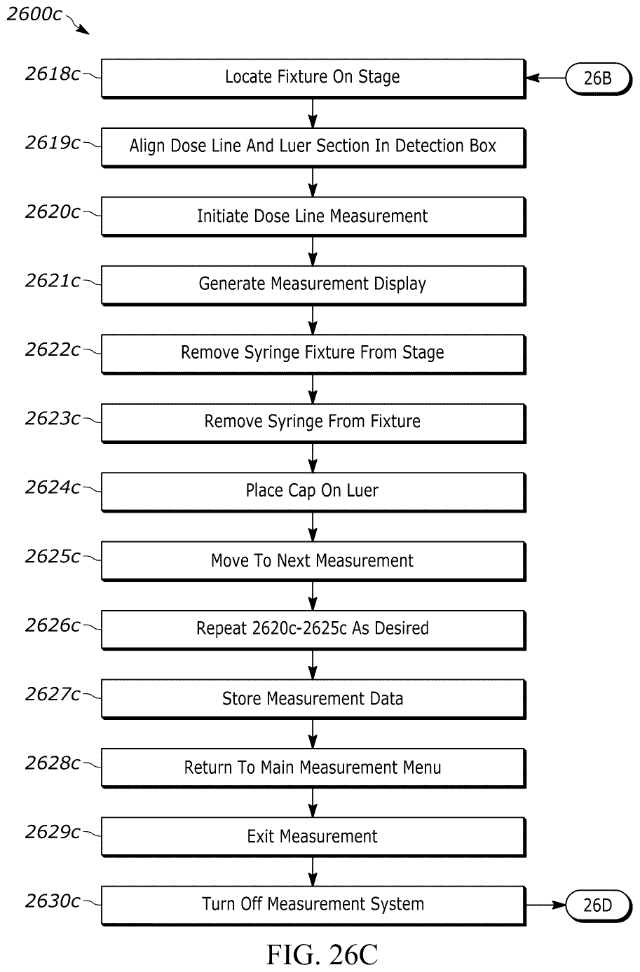

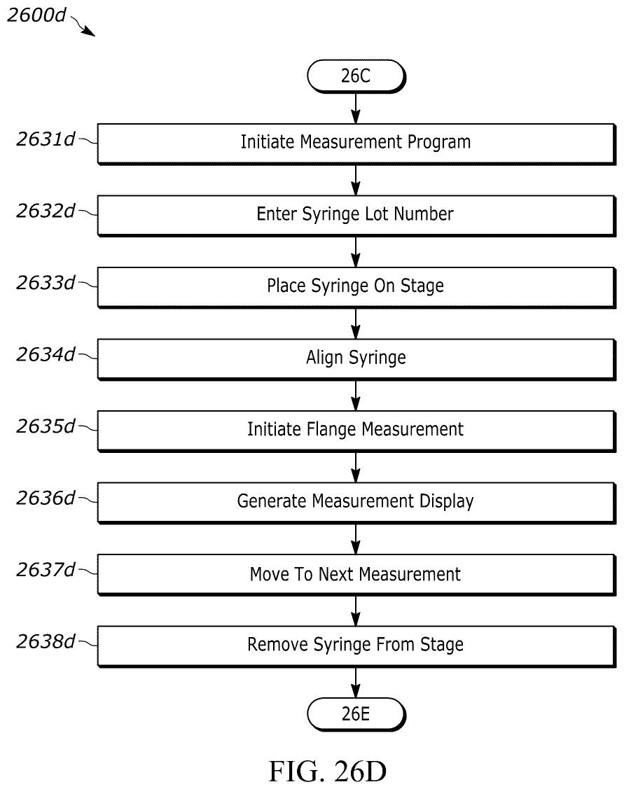

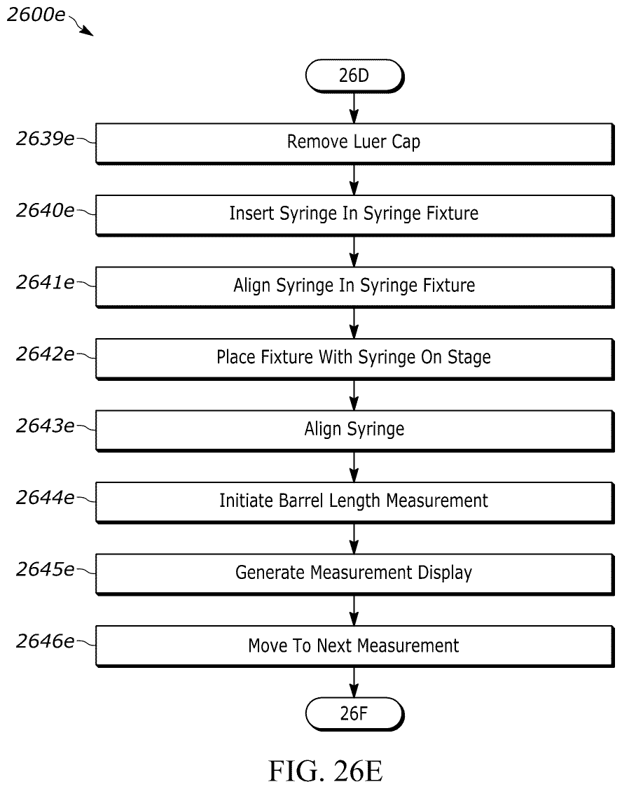

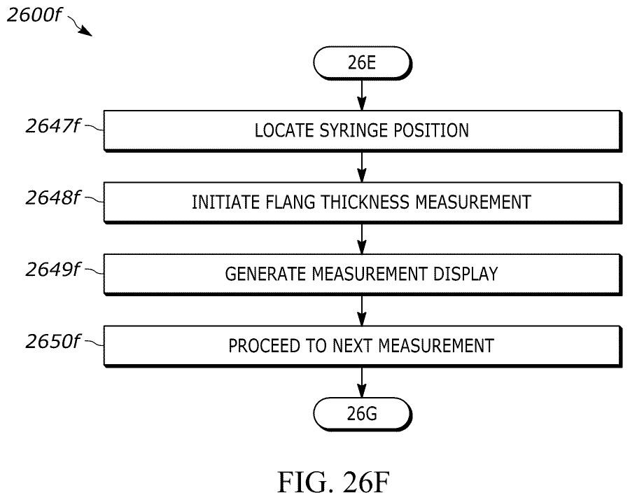

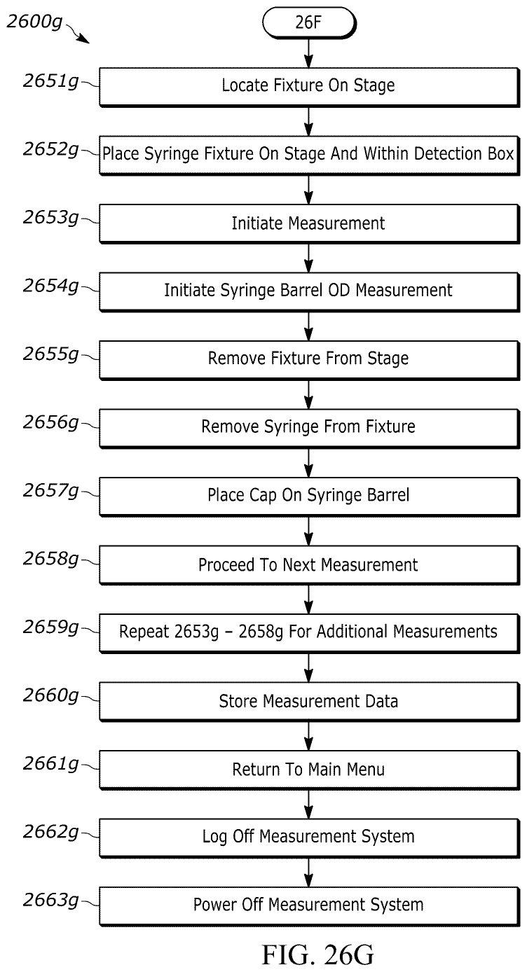

Still referring to FIG. 1A, the system 100 also includes a syringe fixture 145a,d and a light probe 108a. When the syringe fixture 145a,d is placed on the support platform 124, a central syringe axis 126a,d of the syringe 125a,d is aligned with the central image axis 107 in a predetermined orientation. For example, the central syringe axis 126a,d may be aligned parallel to the central image axis 107. In some embodiments, the central syringe axis 126a,d may be coaxially aligned with the central image axis 107. Further details related to measuring geometric dimensions (e.g., a barrel inner diameter) of the syringe 125a,d are described with reference to FIGS. 3-9 and 26A.

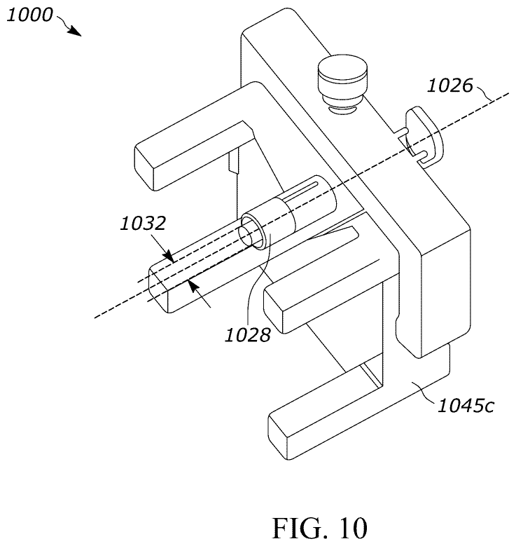

Also illustrated in FIG. 1A is syringe fixture 145c which may be the same as syringe fixture 145a,d with a second planar surface 447c of the syringe fixture 145c proximate the support platform 124. When the syringe fixture 145c is placed on the support platform 124, the central syringe axis 126c of the syringe 125c is aligned with the central image axis 107 in a predetermined orientation. For example, the central syringe axis 126c may be aligned with and possibly intersecting and perpendicular to the central image axis 107. Further details related to measuring geometric dimensions of the syringe 125b are described with reference to FIGS. 10-12 and 26B (e.g., a luer outer diameter); 13, 14, and 26C (e.g., a dose line location); 19-21 and 26E (e.g., a barrel length); 22, 23 and 26F (e.g., a flange thickness); and 24, 25 and 26G (e.g., a barrel outer diameter).

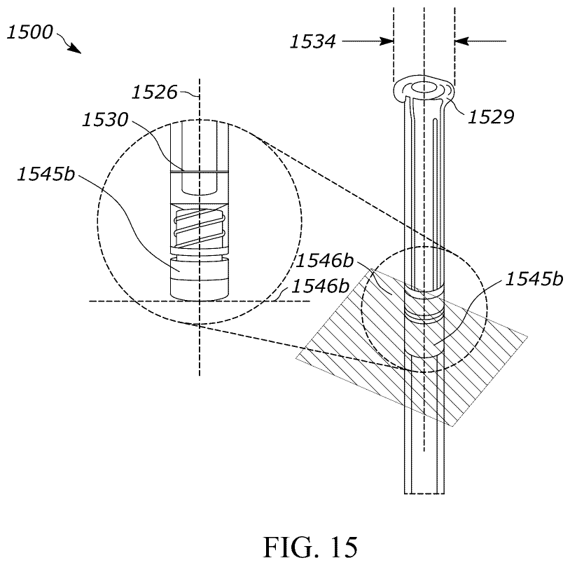

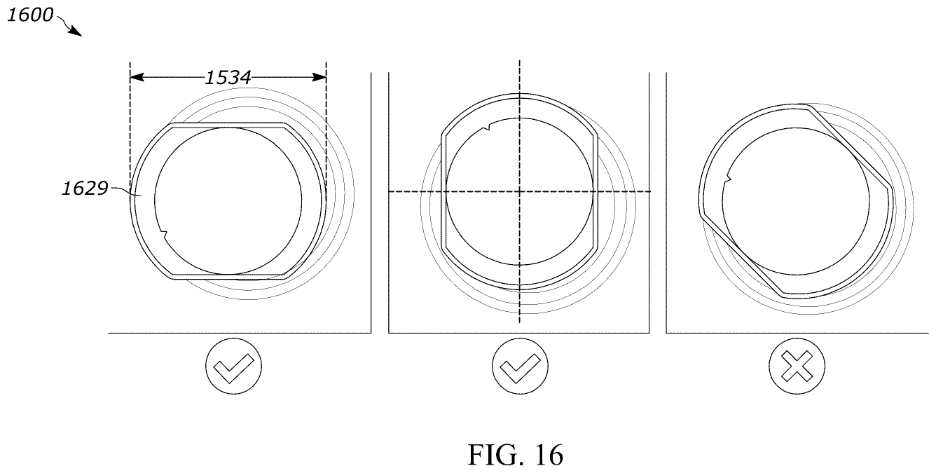

Finally, as depicted in FIG. 1A, the system 100 can also include an alternative form of syringe fixture 145b (e.g., a luer cap). When the syringe fixture 145b is coupled to the syringe 125b and placed on the support platform 124, the central syringe axis 126b of the syringe 125b is aligned with the central image axis 107 in a predetermined orientation. For example, the central syringe axis 126b may be aligned parallel to the central image axis 107. In some embodiments, the central syringe axis 126b may be coaxially aligned with the central image axis 107. Further details related to measuring geometric dimensions (e.g., a flange diameter) of the syringe 125b using syringe fixture 145b are described with reference to FIGS. 15-18 and 26D.

As depicted in FIG. 1B, the disclosed system 100a,b includes a measurement library 110 includes geometric dimension data (e.g., a barrel inner diameter, a luer outer diameter, a dose line location, a flange diameter, a barrel length, a flange thickness, a barrel outer diameter, an overall plunger stopper length, a plunger stopper width, a plunger stopper angular alignment, etc.). The geometric dimension data may include digital image data that is representative of images from which associated geometric dimensions are measured.

In some embodiments, system 100a,b includes two or more computers that are either co-located or remote from each other. In these distributed embodiments, the operations described herein relating to processing unit 117 and memory unit 118 may be divided among multiple processing units and/or memory units, respectively. System 100 may include algorithms (e.g., edge detection algorithms, pixel location algorithms, line orientation algorithms, etc.) for calculating various geometric dimensions.

For example, a measurement system control module 120 and a measurement analysis module 119 may be stored on the memory unit 118 as a set of computer-readable instructions and executed by processing unit 117 to measure geometric dimensions of a syringe and/or a plunger stopper.

Processing unit 117 includes one or more processors, each of which may be a programmable microprocessor that executes software instructions stored in memory unit 118 to execute some or all of the functions of measurement system 105 as described herein. Processing unit 117 may include one or more artificial intelligence (Al) processing units and/or one or more central processing units (CPUs), for example. Alternatively, or in addition, some of the processors in processing unit 117 may be other types of processors (e.g., application-specific integrated circuits (ASICs), field-programmable gate arrays (FPGAs), etc.), and some of the functionality of system 100a,b as described herein may instead be implemented in hardware.

Memory unit 118 may include one or more volatile and/or non-volatile memories. Any suitable memory type or types may be included in memory unit 118, such as read-only memory (ROM), random access memory (RAM), flash memory, a solid-state drive (SSD), a hard disk drive (HDD), and so on. Collectively, memory unit 118 may store one or more software applications, the data received/used by those applications, and the data output/generated by those applications. In other embodiments, memory unit 118 may omit one or more of modules 119, 120 and/or include one or more additional modules. In addition, or alternatively, one, some, or all of modules 119, 120 may be implemented by a different computer system (e.g., a remote server coupled to system 115 via one or more wired and/or wireless communication networks). Moreover, the functionality of any one of modules 119 and 120 may be divided among different software applications and/or computer systems. As just one example, in an embodiment where system 100a,b accesses a web service to train and use one or more geometric dimension measurements models, the software instructions of measurement analysis module 119 may be stored at a remote server. A method of measuring geometric dimensions may be implemented by a processor (e.g., processing unit 117 of FIG. 1B) executing, for example, at least a portion of the measurement analysis module 119 and/or measurement system control module 120.

### Syringe Fixture

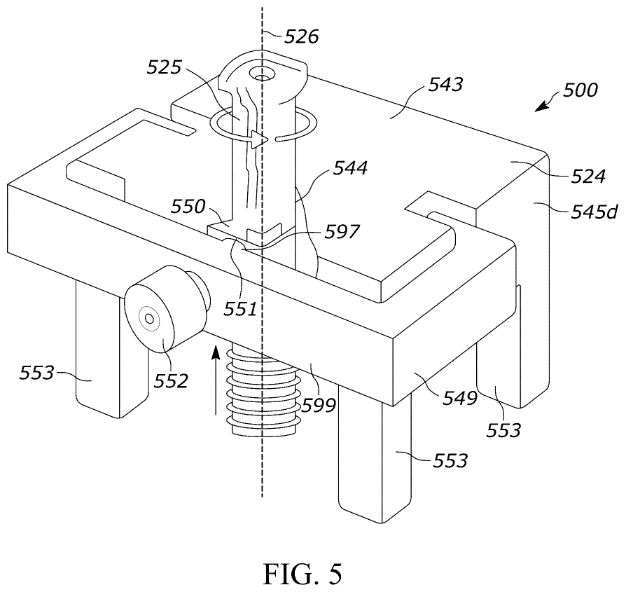

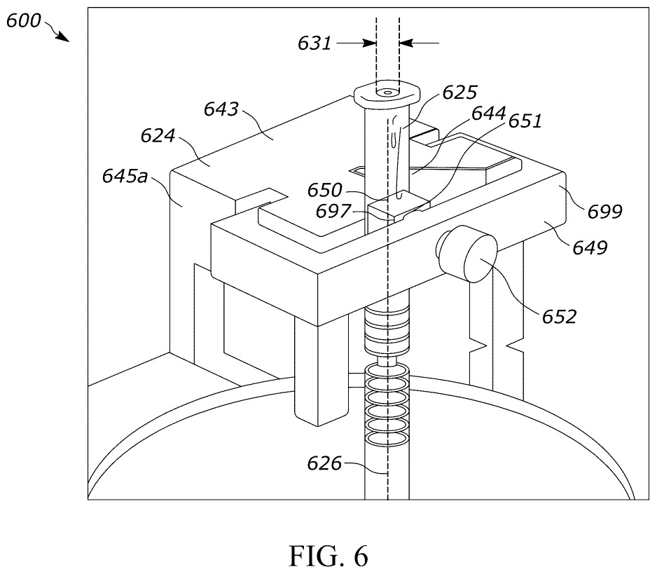

As depicted in FIGS. 3-9, for example, one version of the syringe fixture 345a,d, 445a,d, 545a,d, 645a,d, 745a,d, 845a,d, 945a,d can include a support block 543, 643 (see, FIGS. 5 and 6) and a syringe securing mechanism 599, 699 removably coupled to the support block 543, 643 for securing the syringe in the fixture. In some versions, the support block 543, 643 can include a first color (e.g., black) and the syringe securing mechanism 599, 699 can be a second color (i.e., white) distinct from the first color such that the imaging system 100 can distinguish between and identify edge characteristics, for example, of the two components.

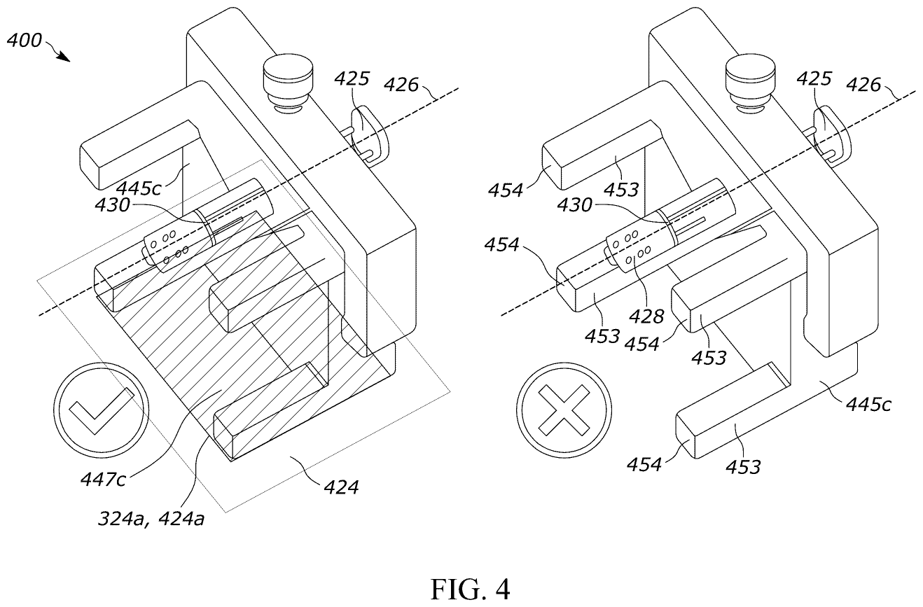

In the disclosed embodiment, the support block 543, 643 comprises a support platform 524, 624 and a plurality of legs 453, 553 extending from the platform 524, 624. In the present version, the support block 543, 643 includes four (4) legs 453, 553 with proximal ends attached to the support platform 524, 624 and distal ends disposed away from the support platform 524, 624. Other versions can include any number of legs suitable to achieve the intended objective. As will be discussed further below, the disclosed configuration of the support block 543, 643 advantageously provides for a fixture that can be used in different orientations for presenting the syringe in different orientations to capture images and collect measurements. To facilitate this objective, and as depicted in FIG. 3, the distal ends of the legs 453, 553 include flat distal end surfaces that collectively define and/or reside within a first planar support surface 324. Additionally, as depicted in FIG. 4, at least one of the plurality of legs 453, 553 defines a side surface disposed perpendicular to the distal end surfaces of the legs 453, 553 that at least partly defines and/or resides in a second planar support surface 424 that is perpendicular to the first planar support surface 324. As illustrated, the first planar support surface 324 includes a first edge 324a and the second planar support surface 424 includes a second edge 424a that conjoins the first edge 324a. With the support block 543, 643 configured in this manner, it can be seen in FIGS. 3 and 4 that when the first planar support surface 324 is placed on the stage 124 of the system 100, the syringe is oriented vertically and when the second planar support surface 424 is placed on the stage 124 of the system 100, the syringe is oriented horizontally. In other embodiments, other orientations are possible within the scope of the present disclosure.

Referring again to FIGS. 5 and 6, the support platform 524, 624 is depicted as generally rectangular in shape (but other shapes are possible) and defines a V-shaped notch 544, 644 in a sidewall thereof. The V-shaped notch 544, 644 is configured to cooperate with the syringe securing mechanism 599, 699 to secure the syringe 325, 425, 525, 625, 725, 825, 925 within the support block 543, 643. In some versions, the V-shaped notch 544, 644 is configured to cooperate with the syringe securing mechanism 599, 699 to center the syringe 325, 425, 525, 625, 725, 825, 925 within the support block 543, 643.

With continued reference to FIGS. 5 and 6, the disclosed version of the syringe securing mechanism 599, 699 includes a syringe clamp 549, 649, a V-shaped clamp 550, 650, a thumb screw 552, 652 and a spring 597, 697. The syringe clamp 549, 649 of the disclosed embodiment is generally C-shaped and slidably coupled into a pair of slots on the sides of the support platform 524, 624. The thumb screw 552, 652 passes through an aperture in the syringe clamp 549, 649 and includes a knurled knob and a shaft that carries the V-shaped clamp on an opposite end from the knurled knob. The spring 597, 697 includes a compression carried on the shaft of the thumb screw 552, 652 as a location between the syringe clamp 549, 649 and the V-shaped clamp 550, 650 such that the spring 597, 697 urges the V-shaped clamp 550, 650 away from the syringe clamp 549, 649 and toward the V-shaped notch 544, 644 on the support block 543, 643. So configured, the spring 597, 697 biases the V-shaped clamp 550, 650 into engagement with a syringe 525, 625 that is present in the V-shaped notch 544, 644 to secure the syringe 525, 625 in position.

### Syringe Measurements

A method 2600a of measuring geometric dimensions (e.g., a barrel inner diameter 631) of the syringe 325, 425, 525, 625, 725, 825, 925 is described with reference to FIGS. 3-9 and 26A. The system 300, 400, 500, 600, 700, 800, 900 may include a syringe fixture 345a,d, 445a,d, 545a,d, 645a,d, 745a,d, 845a,d, 945a,d and a light probe 808a,b. When the syringe fixture is placed on the first planar support surface 324, the central syringe axis 326a,d, 426a,d, 526a,d, 626a,d, 726a,d, 826a,d, 926a,d of the syringe 325, 425, 525, 625, 725, 825, 925 is aligned with the central image axis 107 in a predetermined orientation. For example, the central syringe axis 326, 426, 526, 626, 726, 826, 926 may be aligned parallel to or coaxial with the central image axis 107.

The processing unit 117 may execute the measurement system control module 120 to cause the processing unit 117 to execute a geometric dimension measurement program (block 2601a). The geometric dimension measurement program may be accessed from, for example, a drop-down list as shown in FIG. 2. A lot number 222 in the field labeled “Lot” in the upper right-hand corner of a program screen (block 2602a). A luer cap 145b may be removed from a syringe 125b (block 2603a).

To place a syringe 525, 625 in the fixture 500, 600, for example, a user can pull the thumb screw 552, 652 connected to the syringe clamp 549, 649 thereby compressing the spring 597, 697 and moving the V-shaped clamp 550, 650 away from the V-shaped notch 544, 644. Thumb screw 552, 652 may be connected to the V-shaped clamp 550, 650, and designed to be used as a grip to pull on the V-shaped clamp 550, 650 (block 2604a). The syringe 425 may be rotated around a central syringe axis 426 within the fixture 445c so the dose line cutout 430 is facing down as shown in FIG. 4 (block 2605a). A user may pull on the thumb screw 552, 652 and lower the syringe barrel until it touches the flat surface (e.g., support platform 324, and may rotate the syringe 325 to ensure a flange 1529 width is horizontal with any edge as shown in FIG. 5. A flange parallelism with the edge may be approximated (block 2606a).

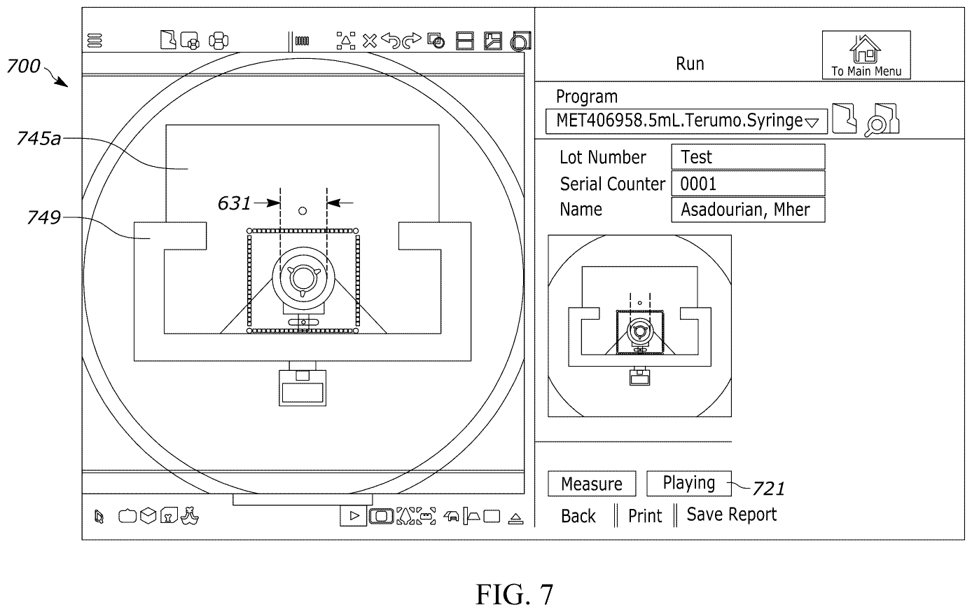

The fixture 645a is then placed on the stage 124 so that that, for example, the fixture 645a stands on the distal end surfaces of the legs 553 and the thumb screw 652 is facing as shown in FIG. 6 (block 2607a). A flange section of the syringe may be, for example, placed with a detection box 721 of a user interface display 700 as shown in FIG. 7 (block 2608a). A “Measure” button as shown in FIG. 7 may be pressed (block 2609a).

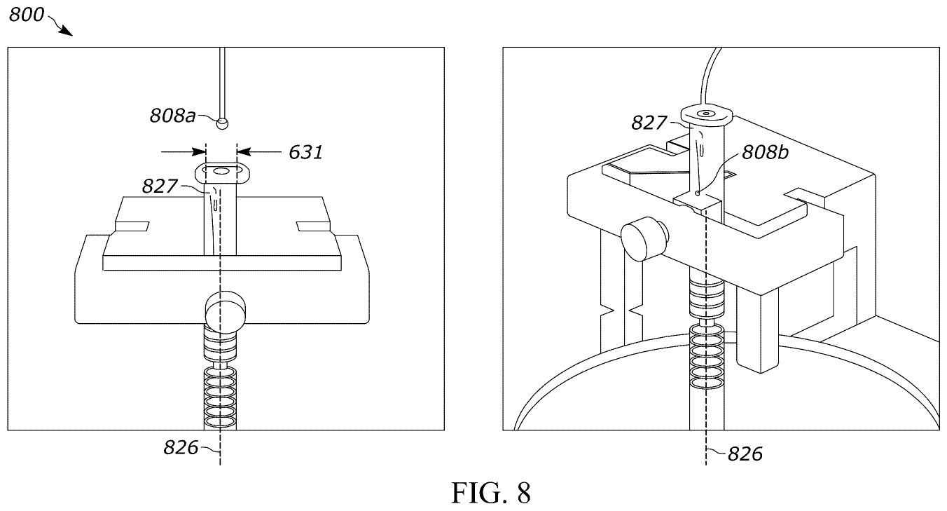

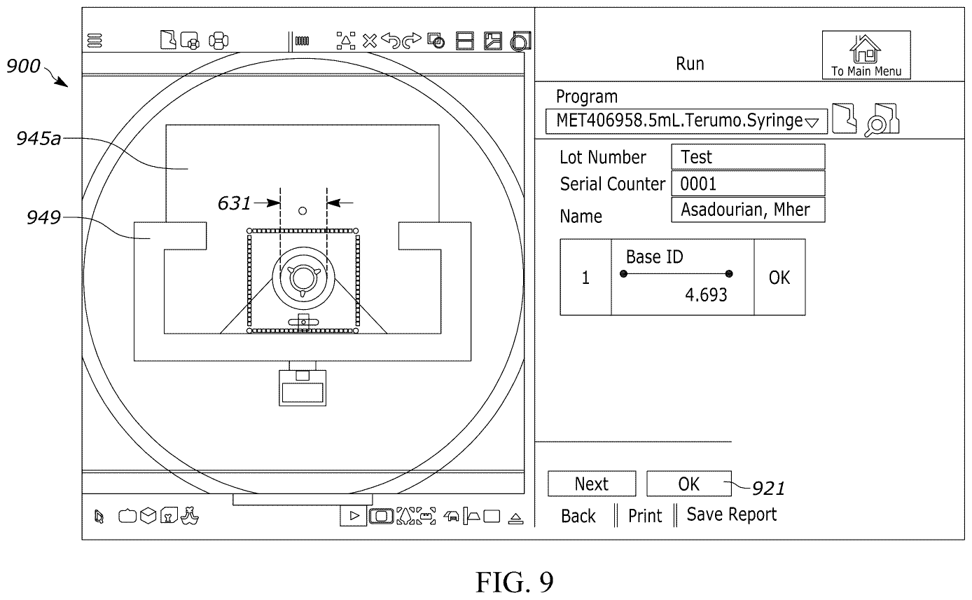

The processing unit 117 may execute the measurement device control module 120 to, for example, cause the light probe 808a arm to swing towards the stage 124 from the side and will go in-side the barrel 808b to take a barrel ID measurement as shown in FIG. 8 (block 2610a). Once the probe takes all the barrel ID measurements, the maximum barrel ID value may be display on the right side of the screen as shown in FIG. 9 (block 2611a). A “Next” may be selected as shown in FIG. 9 (block 2612a), and the geometric dimension measurement program will proceed to the next measurement.

A method 2600b of measuring geometric dimensions (e.g., a luer outer diameter 1032) of the syringe 125b is described with reference to FIGS. 10-12 and 26B. The geometric dimension measurement system 1000, 1100, 1200 includes a syringe fixture 1045c, 1145c, 1245c. The syringe fixture 1045c, 1145c, 1245c may be similar to, for example, the syringe fixture 145b. As de-scribed in detail with reference to FIGS. 3, 4 and 10-14, the syringe fixture 1045c, 1145c, 1245c may be the same as syringe fixture 145b with a second planar surface 447c of the syringe fixture 145c proximate the support platform 124. When the syringe fixture 1045c, 1145c, 1245c is placed on the second planar support surface 424, the central syringe axis 1026, 1126, 1226 of the syringe 1025, 1125, 1225 is aligned with the central image axis 107 in a predetermined orientation. For example, the central syringe axis 1026, 1126, 1226 may be aligned perpendicular to the central image axis 107.

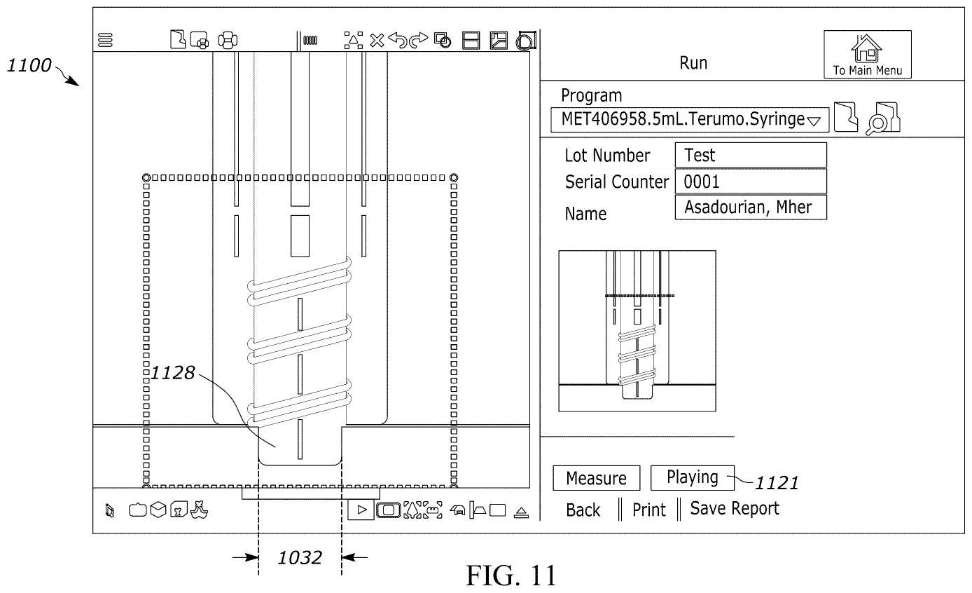

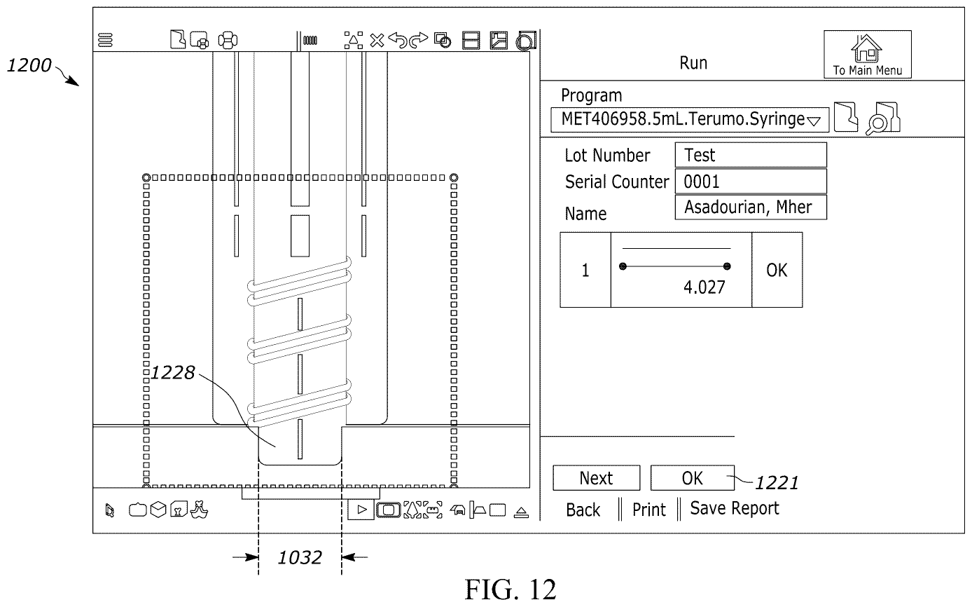

The fixture 1045c, 1145c, 1245c may be rotated on the stage 124 without touching a syringe barrel 1927 with thumb screw facing up and a luer cone pointing as shown in, for example, FIG. 10 (block 2613b). A luer section 1128 of the barrel is within the detection box as shown in FIG. 11 (block 2614b). A user may initiate the “Measure” button as shown in FIG. 11 (block 2615b). The geometric dimension measurement program may be executed, and the measurement will display on the right side of the screen as shown in FIG. 12 (block 2615b). A “Next” icon may be pressed as shown in FIG. 12 (block 2617b), and the program will proceed to the next measurement.

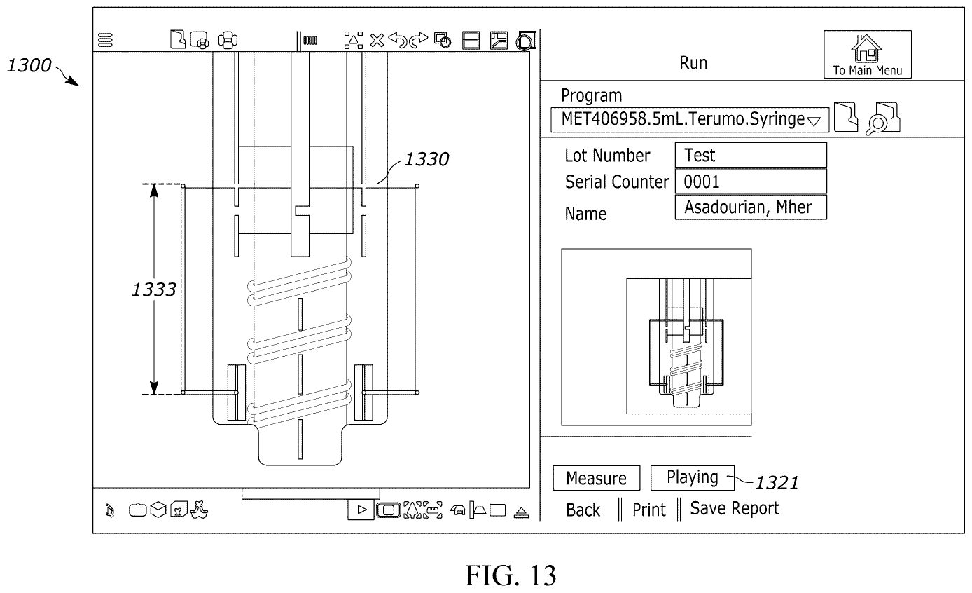

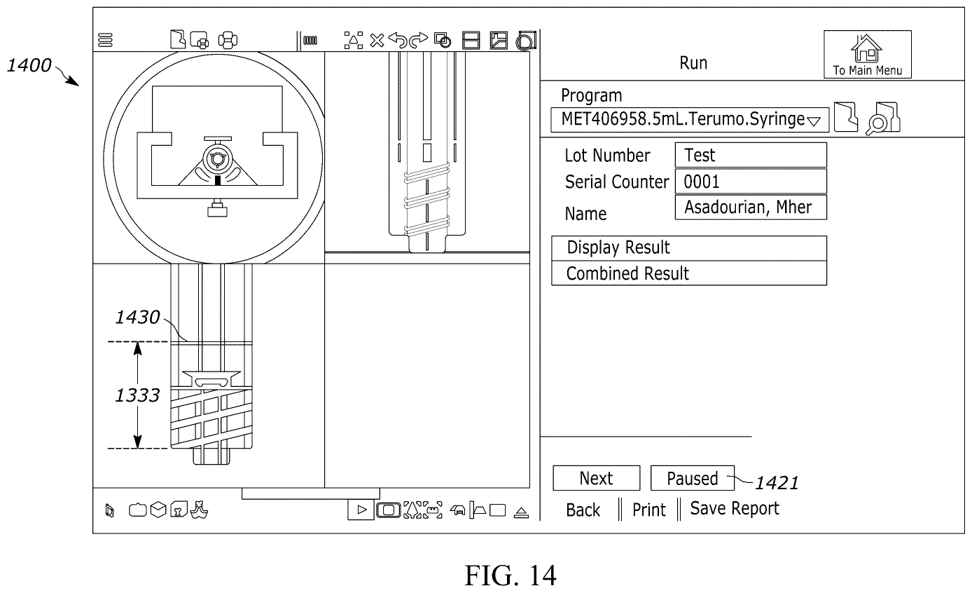

A method 2600c of measuring geometric dimensions (e.g., a dose line location 1333) of the syringe 1325, 1425 is described with reference to FIGS. 13, 14, and 26C. The system 1300, 1400 includes a syringe fixture 1345c, 1445c. The syringe fixture 1345c, 1445c may be similar to, for example, the syringe fixture 145b. As described in detail with reference to FIGS. 3, 4 and 10-14, the syringe fixture 1345c, 1445c may be the same as syringe fixture 145b with the second planar surface 447c of the syringe fixture 145c proximate the support platform 124. When the syringe fixture 1345c, 1445c is placed on the support platform 124, the central syringe axis 1326, 1426 of the syringe 1325, 1425 is aligned with the central image axis 107 in a predetermined orientation. For example, the central syringe axis 1326, 1426 may be aligned perpendicular to the central image axis 107 (block 2618c). Fixture's placement doesn't need to be changed or adjusted for this measurement. A user may orient the dose line and the luer section of the barrel as shown in FIG. 13 (block 2619c). A “Measure” button as shown in FIG. 13 may be selected (block 2620c). The geometric dimension measurement program will be executed and may display all the critical measurements as shown in FIG. 14 (block 2621c).

The fixture 1345c, 1445c may be removed from the stage 124 (block 2622c). The syringe may be removed from the fixture (block 2623c). The luer cap may be placed back on the barrel (block 2624c). A user can review the results of each program by selecting the program from the drop-down menu under Display Results shown in FIG. 14. A “Next” icon may be selected on the screen as shown in FIG. 14 (block 2625c). Blocks 2620c-2625c may be repeated for any additional luer syringes that need to be tested for critical dimensions (block 2626c). Measurement data (e.g., geometric dimension measurements) may be stored (block 2627c). A user may return to a main menu screen by clicking “To Main Menu” button (block 2628c). A user may exit a measurement system control module (block 2629c). A user may log out of the geometric measurement system (block 2630c).

A method 2600d of measuring geometric dimensions (e.g., a flange diameter 1534) of the syringe 125b is described with reference to FIGS. 15-18 and 26D. The system 1500, 1600, 1700, 1800 includes a syringe fixture 1545b, 1645b, 1745b, 1845b (e.g., a luer cap). When the syringe fixture 1545b, 1645b, 1745b, 1845b is placed on the support platform 124, the central syringe axis 1526, 1626, 1726, 1826 of the syringe 1525, 1625, 1725, 1825 is aligned with the central image axis 107 in a predetermined orientation. For example, the central syringe axis 1526, 1626, 1726, 1826 may be aligned parallel to the central image axis 107.

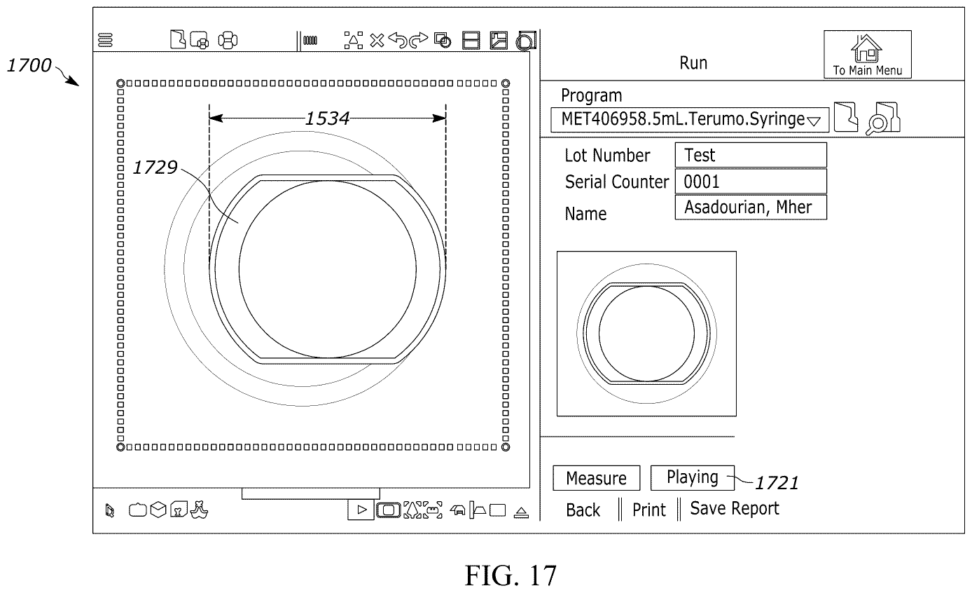

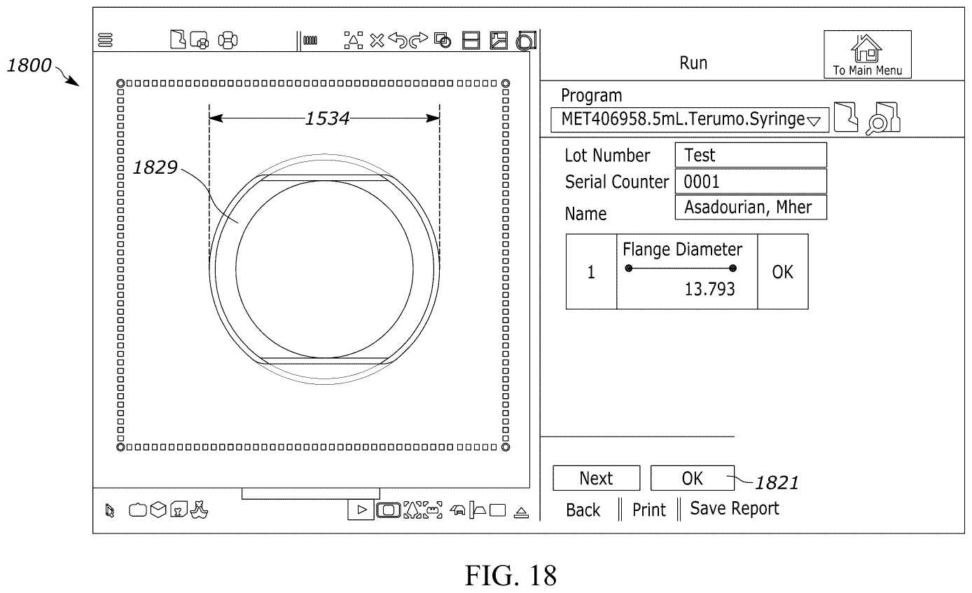

The processing unit 117 may further execute the measurement system control module 120 to, for example, cause the processing unit 117 to load the geometric dimension measurement program as shown in FIG. 2 (block 2631d). A user may enter the lot number 222 in the field labeled “Lot” (block 2632d). The syringe fixture 1545b, 1645b, 1745b, 1845b is placed on the stage 124 vertically, flange side up as shown in FIG. 15 (block 2633d). The syringe barrel is oriented on the stage 124 so that the flange cutout is either horizontal or vertical to the stage (block 2634d). A “Measure” button may be selected as shown in FIG. 17 (block 2635d). The processing unit 117 may execute the measurement analysis module 119 to, for example, cause the processing unit 117 to generate a measurement display on the right side of the screen 1821 as shown in FIG. 18 (block 2636d). A user may select “Next” as shown in FIG. 18 (block 2637d), and the geometric dimension measurement program will proceed to the next measurement (block 2638d).

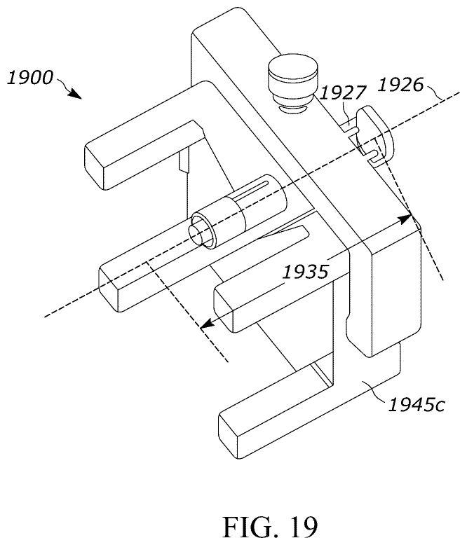

A method 2600e of measuring geometric dimensions (e.g., a barrel length 1935) of the syringe 1925, 2025, 2125 is described with reference to FIGS. 19-21 and 26E. The system 1900, 2000, 2100 includes a syringe fixture 1945c, 2045c, 2145c. The syringe fixture 1945c, 2045c, 2145c may be similar to, for example, the syringe fixture 145b. As described in detail with reference to FIGS. 3, 4 and 10-14, the syringe fixture 1945c, 2045c, 2145c may be the same as syringe fixture 145b with a second planar surface 447c of the syringe fixture 145c proximate the support platform 124. When the syringe fixture 1945c, 2045c, 2145c is placed on the support platform 124, the central syringe axis 1926, 2026, 2126 of the syringe 1925, 2025, 2026 is aligned with the central image axis 107 in a predetermined orientation. For example, the central syringe axis 1926, 2026, 2126 may be aligned perpendicular to the central image axis 107.

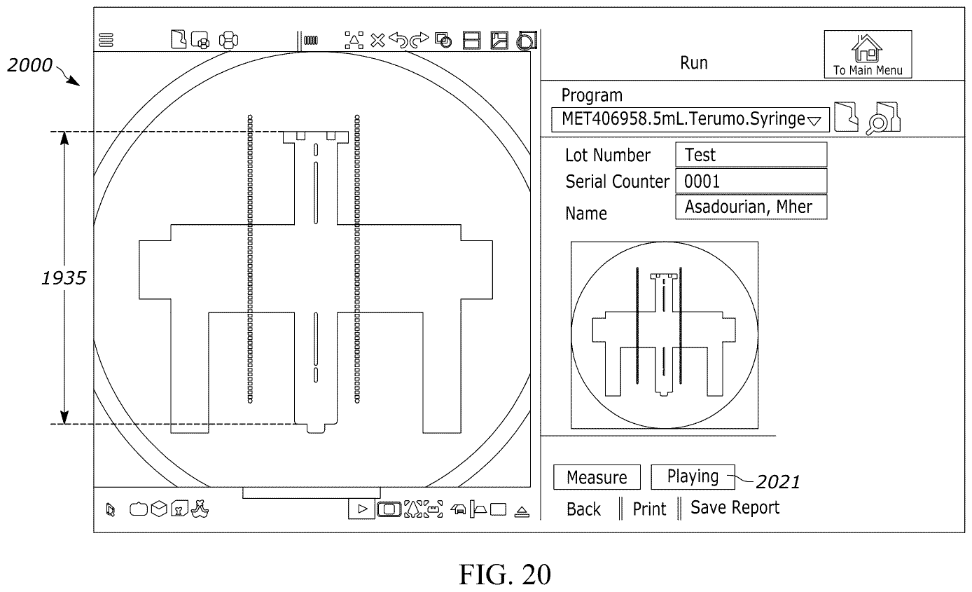

A luer cap may be removed (e.g., unscrewed) (block 2639e). The syringe 1925, 2025, 2125 may be placed in the syringe fixture 1945c, 2045c, 2145c as shown in FIG. 3 (block 2640e). The thumb screw may be connected to the clamp and may be designed to be used as a grip to pull on the syringe barrel clamp and is not designed to be twisted (block 2641e). The thumb screw may be pulled and lower the syringe barrel until it hits the flat surface and rotate the barrel to ensure the flange width is horizontal with any edge of the syringe fixture 1945c, 2045c, 2145c as shown in FIG. 4 (block 2642e). The flange parallelism with the edge can be approximated (block 2643e). The fixture 1945c, 2045c, 2145c is placed on the stage without touching the syringe barrel, with thumb screw facing up and the Luer cone pointing as shown in FIG. 19. A user may align the fixture as shown in FIG. 20.

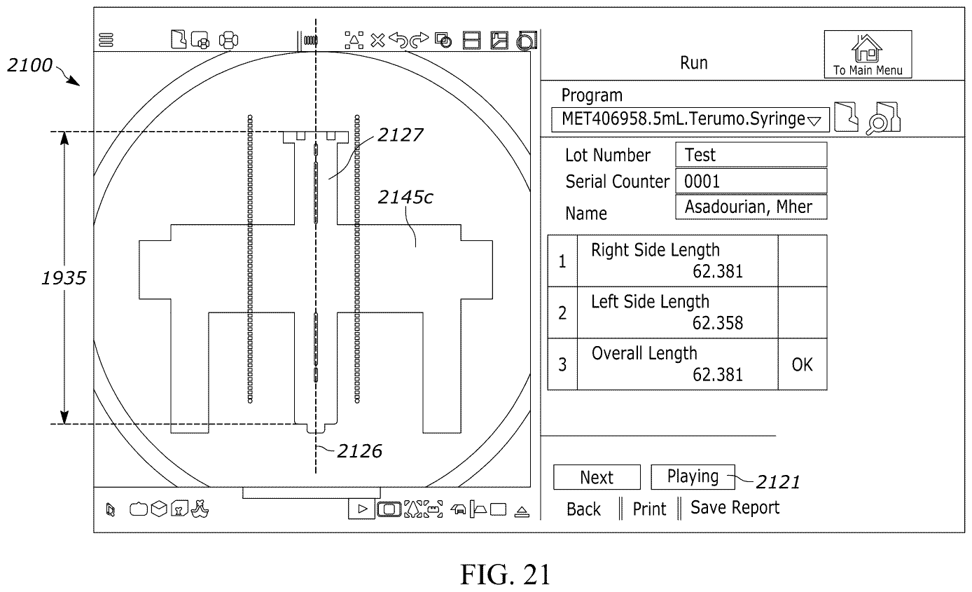

A “Measure” button may be selected as shown in FIG. 20 (block 2644e). The geometric dimension measurement program will be executed, the program will take two measurement and display an average on the right side of the screen as shown in FIG. 21 (block 2645e). The user may select a “Next” icon as shown in FIG. 21 (block 2646e), and the program will proceed to the next measurement.

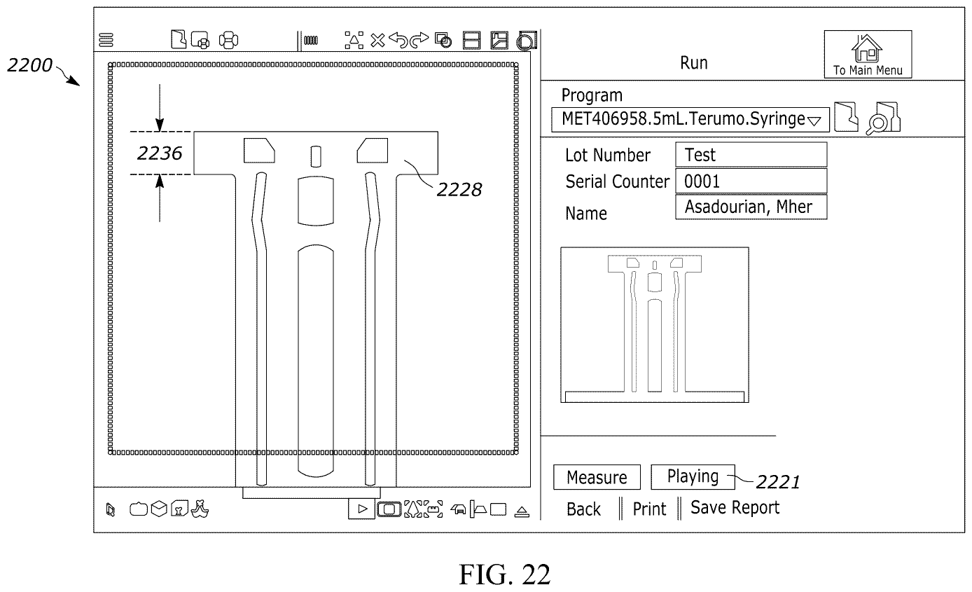

A method of measuring geometric dimensions (e.g., a flange thickness 2236) of the syringe 2225, 2325 is described with reference to FIGS. 22, 23 and 26F. The system 2200, 2300 includes a syringe fixture 145c. The syringe fixture 2245c, 2345c may be similar to, for example, the syringe fixture 145b. As described in detail with reference to FIGS. 3, 4 and 10-14, the syringe fixture 2245c, 2345c may be the same as syringe fixture 145b with a second planar surface 447c of the syringe fixture 145c proximate the support platform 124. When the syringe fixture 2245c, 2345c is placed on the support platform 124, the central syringe axis 2226 2326 of the syringe 2225, 2325 is aligned with the central image axis 107 in a predetermined orientation. For example, the central syringe axis 2226, 2326 may be aligned perpendicular to the central image axis 107.

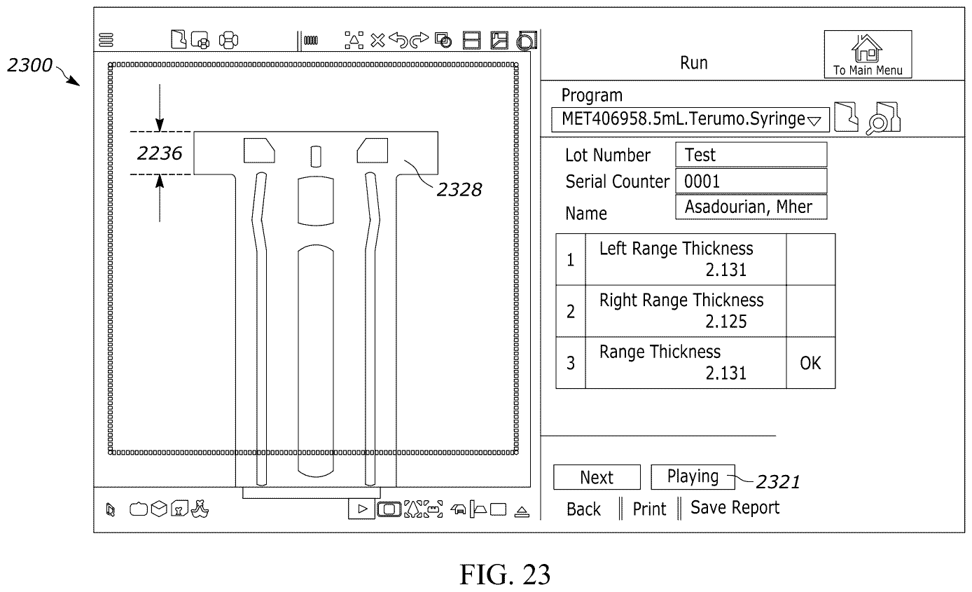

The fixture is placed on the stage 124 and position the flange section 2228 is within the detection box as shown in FIG. 22 (block 2647f). A “Measure” button may be selected as shown in FIG. 22 (block 2648f). The processing unit 117 may executed the measurement system control module 120 to cause the processing unit to initiate the geometric dimension measurement program. The program will take two measurement and display the maximum value on the right side of the screen as shown in FIG. 23 (block 2649f). A “Next” icon may be selected as shown in FIG. 23 (block 2650f) and the program will proceed to the next measurement.

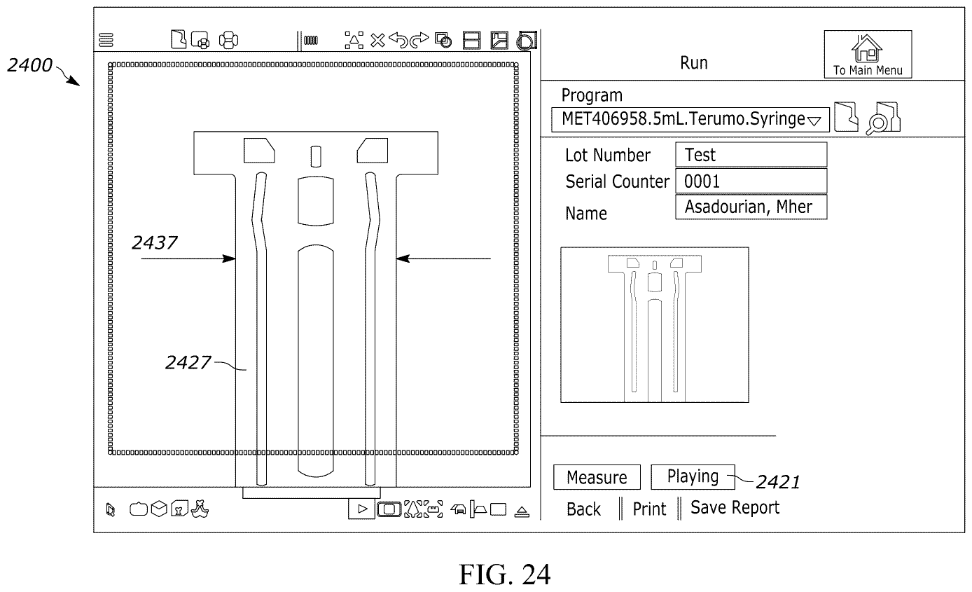

A method 2600g of measuring geometric dimensions (e.g., a barrel outer diameter 2437) of the syringe 2425, 2525 is described with reference to FIGS. 24, 25 and 26G. The system 2400, 2500 includes a syringe fixture 2445c, 2545c. The syringe fixture 2445c, 2545c may be similar to, for example, the syringe fixture 145b. As described in detail with reference to FIGS. 3, 4 and 10-14, the syringe fixture 2445c, 2545c may be the same as syringe fixture 145b with a second planar surface 447c of the syringe fixture 145c proximate the support platform 124. When the syringe fixture 2445c, 2545c is placed on the support platform 124 (or, the central syringe axis 2426, 2526 of the syringe 2425, 2525 is aligned with the central image axis 107 in a predetermined orientation. For example, the central syringe axis 2426, 2526 may be aligned perpendicular to the central image axis 107.

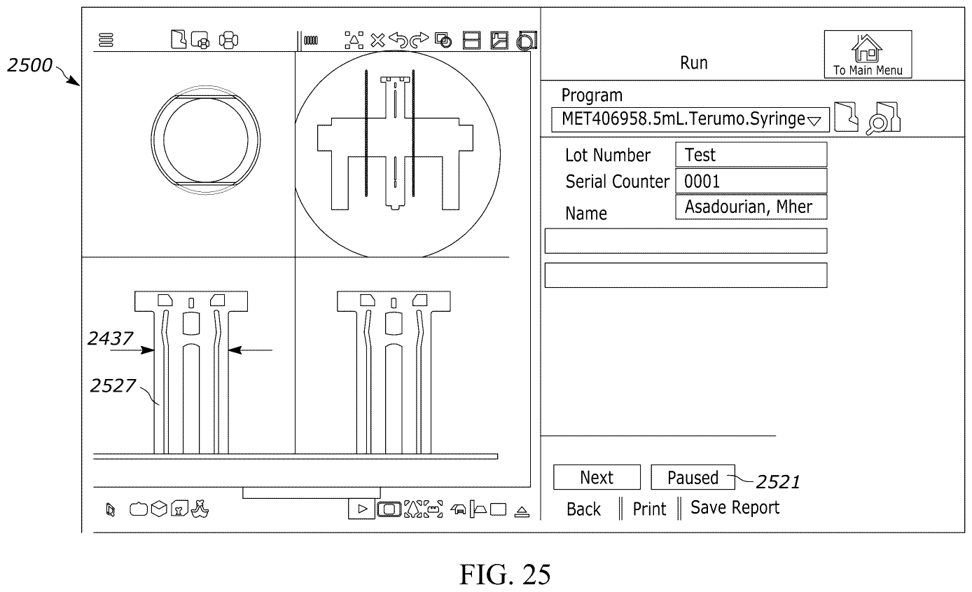

The fixture placement doesn't need to be changed or adjusted for this measurement (block 2651g). The fixture should be within the detection box on the screen as shown in FIG. 24 (block 2652g). The “Measure” button may be selected as shown in FIG. 24 (block 2653g). The processing unit 117 may execute the measure system control module 120 to, for example, cause the processing unit 117 to generate a display 2421 of all the Major B measurements of 4 programs as shown in FIG. 25 (block 2654g). The fixture may be removed from the stage 124 (block 2655g). The syringe from the fixture (block 2656g). The luer cap may be screwed back on the barrel (block 2657g). The user can review the results of each program by selecting the program from the drop-down menu under Display Results shown in FIG. 25. The “Next” icon may be selected on the screen as shown in FIG. 25 (2658g). Blocks 2653g-2658g may be repeated for any additional luer syringes that need to be tested for majorB dimensions (block 2659g). Geometric dimension measurement data may be stored or as applicable (block 2660g). A user may return to the Main Menu screen by clicking “To Main Menu” button on the top right-hand corner (block 2661g). The user may log out of the system (block 2662g). The user may power off the system if necessary, by pushing the ‘Power’ button (block 2663g).

Three (3) critical dimensions and four (4) major B dimensions may be measured. Raw data out-puts may include all the measurement, a mean and standard deviation for each dimension for critical distance (K) calculation.

### Plunger Stopper Fixture

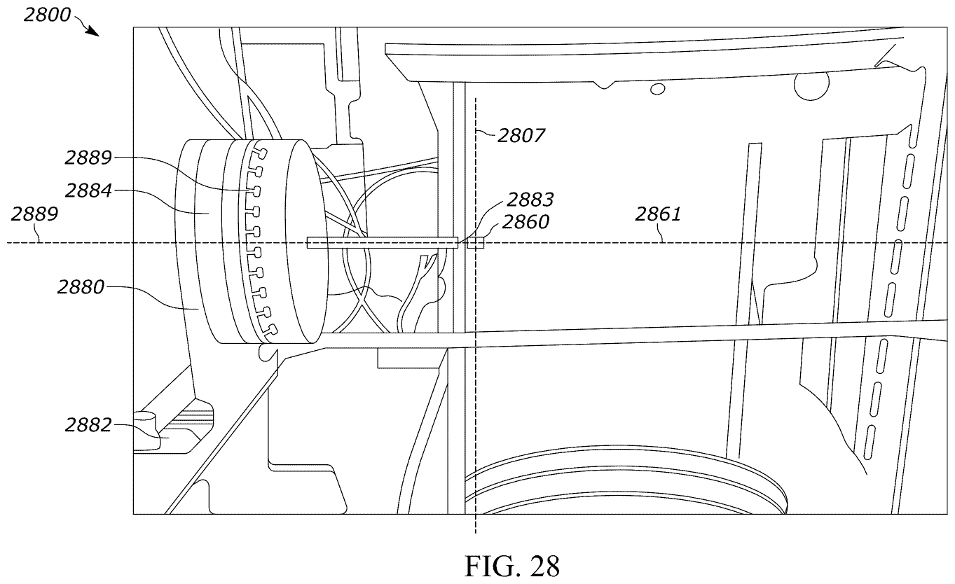

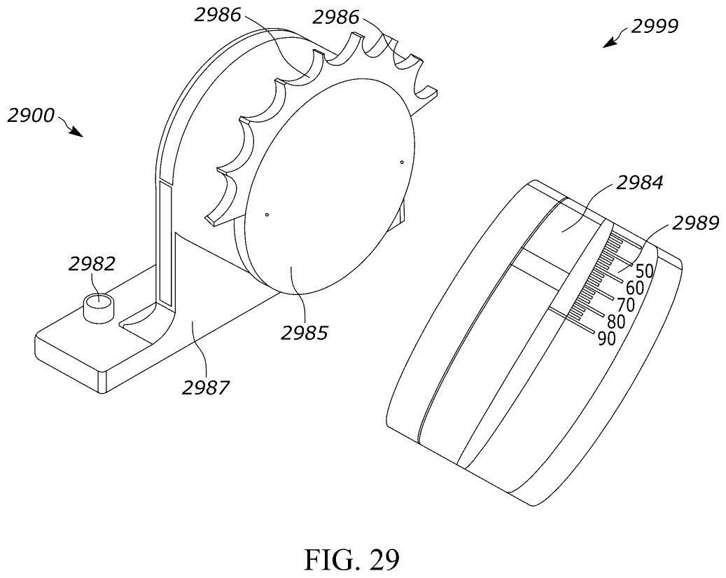

As mentioned herein the present application also discloses methods and systems for measuring plunger stoppers with the imaging system 200. To assist with this process, a plunger stopper fixture 180 is provided. The plunger stopper fixture 180 is depicted in more detail in FIGS. 28 and 29 and includes a base 2987 and a plunger stopper rotation mechanism 2999 (seen in FIG. 28) coupled to the base 2987 for holding and rotating the plunger stopper to be measured. The base 2987 includes a generally flat supporting structure that can be screwed or otherwise secured to the stage 124 of the imaging system 100, as shown in FIG. 28. Still referring to FIG. 28, the plunger stopper rotation mechanism 2999 includes a dial 2884 and a rod 2888. The dial has a dial axis 2889 about which it rotates. The rod 2888 has a proximal end coupled to the dial 2884 and extends along the dial axis 2889 and too rotates about the dial axis 2889 when the dial 2884 rotates. The rod 2888 further includes a distal end disposed away from the dial 2884. The distal end of the rod 2888 defines a plunger stopper retaining tip 2883 for coupling a plunger stopper to the fixture. In some versions, the plunger stopper retaining tip 2883 can include a male thread for being threaded into a cavity of a plunger stopper, for example. Other configurations for attaching the plunger stopper to the distal end of the rod 2888 are possible. AS can be seen in FIG. 29, the dial 2884 includes a rotational angle gauge (e.g., rotational angle indices) on the outside thereof. In some versions, the dial 2884 can include nine (9) rotational indices each representing 20 degree rotations thereby totally 180 degrees of rotation for capturing nine (9) images and determining associated dimensions, as will be described below. Other embodiments can include any number of indices and rotational positions.

### Plunger Stopper Measurements

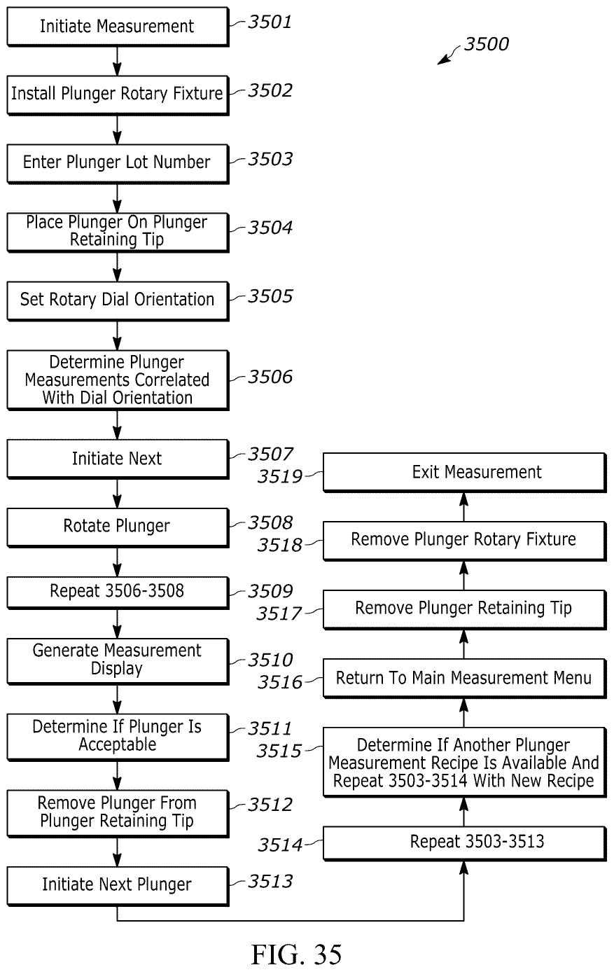

A method 3500 of measuring geometric dimensions (e.g., an overall length 3063, a width 3062, an angular alignment 3464, etc.) of a plunger stopper 160 is described with reference to FIGS. 27-35. The system 2700, 2800, 2900, 3000, 3100, 3200, 3300, 3400, 3500 includes the plunger stopper fixture 180 having the base 2987 (shown in FIG. 29) for being secured to a stage 124 of the imaging system, as shown in FIGS. 1 and 28, for example. When the imaging device attachment 182, 2982 of the plunger stopper fixture 180 is attached to the plunger stopper fixture attachment 101, the central plunger stopper axis 181, 2861 of the plunger stopper 160 is aligned with and possibly intersecting the central image axis 107. For example, with reference to FIG. 35, the central plunger stopper axis 181, 2861 of the plunger stopper 160 may be aligned perpendicular to and possibly intersecting the central image axis 107 in order to initiate a measurement procedure (block 3501).

The plunger stopper fixture 180 may be mounted to the stage 124 using, for example, with appropriate threaded holes and two (2) M4 socket head screws (block 3502). For example, the two (2) M4 socket head screws may be hand tightened so that the fixture 180 is rigid on the stage as shown in FIG. 10. Any screws with M4 thread and 10-13 mm length can be used to install the plunger stopper fixture 180. The Keyence program has three (3) possible outcomes: i. The measurement result is displayed on the Keyence screen with “OK” on the right. This means the measurement of this critical dimension is within the specification limit. In this case, refer to Test Method section for appropriate data recording requirement; ii. The measurement result is displayed on a screen with “NG” on the right. This means the measurement of this critical dimension is outside of specification limit. In this case, refer to Test Method section for appropriate data recording requirement; and iii. The result is displayed with “Fail” on the right. This means the pattern was not properly detect-ed or pattern recognition failed. This does NOT necessarily mean that the sample is out of specification. A user may input the plunger stopper lot number 222 in the Lot Number field, located in the top right corner of the screen 200 (block 3503). A cavity in the back side of the plunger stopper may be placed onto the plunger stopper retaining tip 2883 on the distal end of the rod 2888 (block 3504). This can include threading the plunger stopper onto the tip, inserting the plunger stopper onto the tip with a friction fit, or other means. A user may ensure the plunger stopper 2860 has been fully seated on the retaining tip 2883 by gently tapping on the tip of plunger stopper.

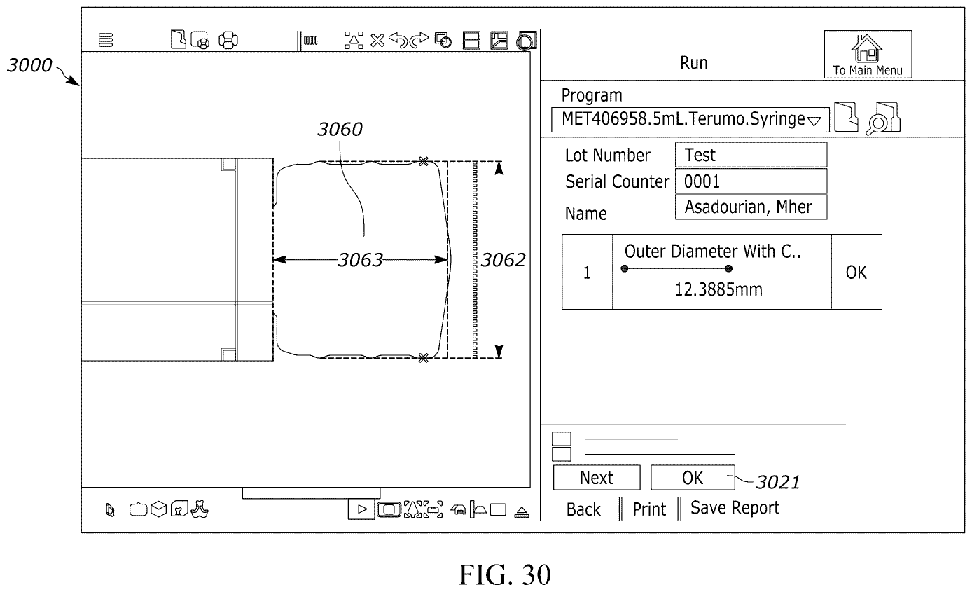

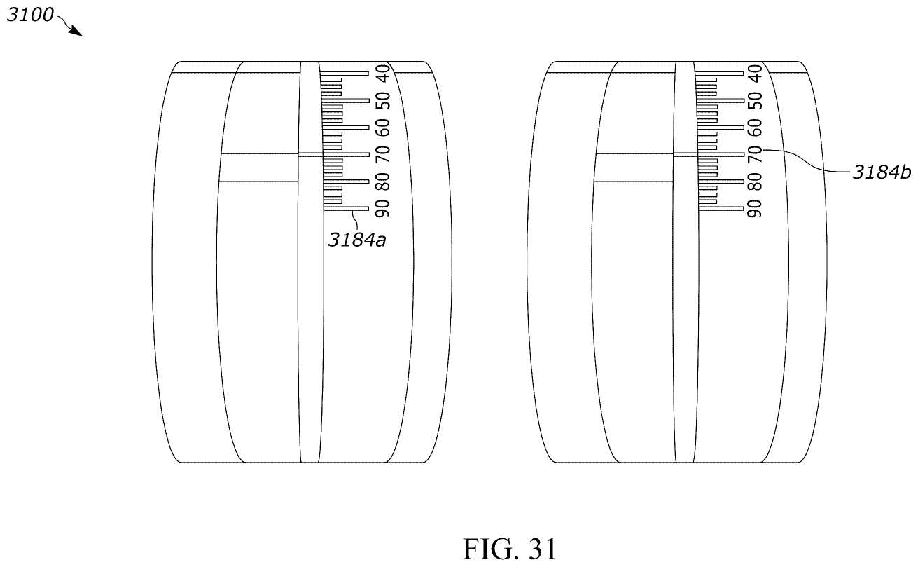

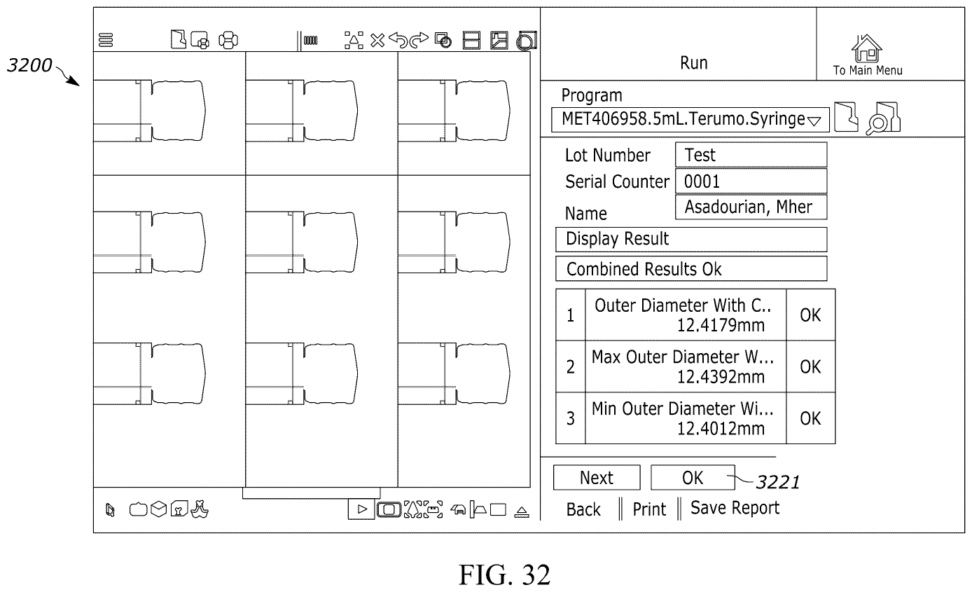

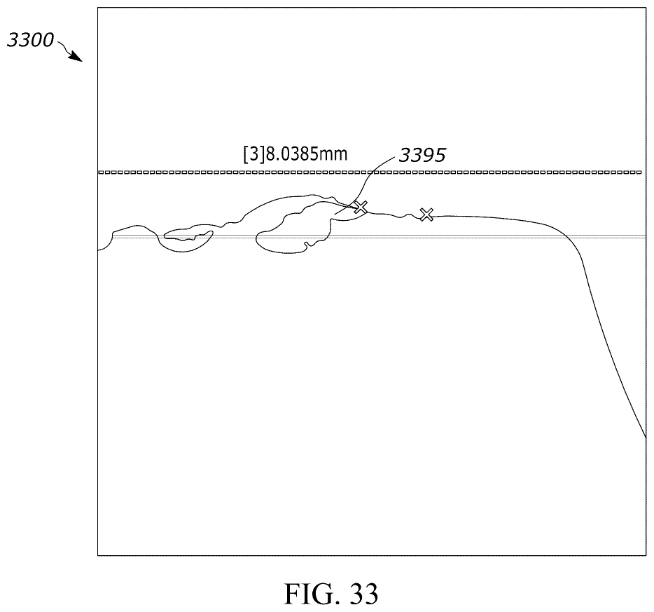

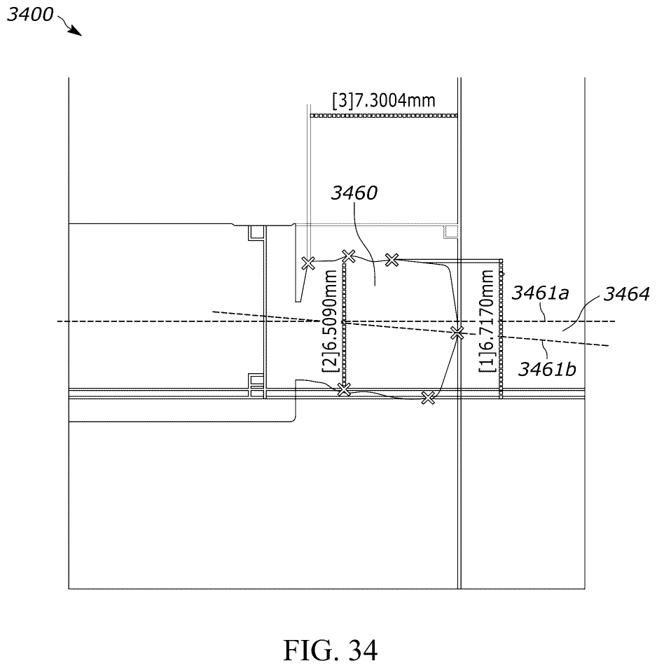

A user may verify that the plunger stopper is parallel to the stage by ensuring that the general position of the plunger stopper does not change upon fixture rotation as shown in FIG. 28 for a visual representation. Using the markers 2889, 2989, 3189a, 3189b on the dial 2884, 2984, 3184a, 3184b as reference, set the rotary dial of the fixture to either of the 90° mark as shown in FIGS. 28, 29 and 31 (block 3505). A user may select the “Measure” button (block 3506). Once the measurement is complete (blocks 3507-3509), the plunger stopper dimensions are displayed (block 3510). FIG. 30 shows an example display 3100. The user may select a “Next” button on the screen to move onto the next measurement (block 3507). Using the markers on the dial as reference, the user may rotate the dial on the fixture by 20° as shown in FIG. 31. Blocks 3506-3508 for a total of nine (9) 20° increment angles (block 3509). All the rotations are in the same direction. After measurement from the nine (9) angles are complete, the program is complete and will display an overview of all nine (9) measurements and three (3) results for each dimension: the average, the maximum, and the minimum, all of which are calculated from the nine (9) measurements. Programs may have one (1) or more dimensions depending on the plunger stopper and classification of its defects. FIG. 32 shows an example display 3200 for the plunge. An average of nine (9) measurements for each dimension is displayed as the name of that dimension without any word preceding or following, for example “OUTER DIAMETER WITH COATING” or “OVERALL LENGTH”. A user may click on the image of each measurement to show the image of that measurement in full screen 3300, 3400 of FIGS. 33 and 34. FIG. 33 depicts dust/hair/debris 3395. A user may click on the image on screen 3300, 3400 to return to the overview. The measured plunger stopper may be removed and store or discard per appropriate procedures (block 3512). A user may click the “Next” button to start the program again (block 3519). Note that the serial counter will roll to the next integer. Blocks 3503-3513 may be repeated until all plunger stoppers are tested (block 3514). A user may verify how many recipe(s) exist for the sample type, if more than 1 recipe repeat blocks 3503-3513 for every recipe. If only one recipe exists for the sample type proceed (block 3515) and return to the Main Menu screen by clicking “To Main Menu” button on the top right-hand corner of FIG. 34 (block 3516). The plunger stopper retaining tip 2883 may be removed from the rod 2888 by unscrewing (block 3517). The two (2) M4×10 socket head screws may be un-screwed, and the plunger stopper fixture 180 may be removed (block 3518). A user may log out and/or shut down the system if necessary (block 3519).

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A syringe fixture configured to align a central syringe axis of a syringe with respect to a central image axis of an imaging system, the syringe fixture comprising:
a support block defining a V-shaped notch, a first planar support surface, and a second planar support surface,
the first planar support surface having a first edge,
the second planar support surface having a second edge, wherein at least a portion of the second edge conjoins at least a portion of the first edge, and wherein the second planar support surface is perpendicular to the first planar support surface; and
a syringe securing mechanism coupled to the support block and configured to secure a syringe in a fixed position in the V-shaped notch such that the syringe occupies a first orientation when the support block is positioned on the first planar support surface and a second orientation perpendicular to the first orientation when the support block is positioned on the second planar support surface.

2. The syringe fixture of claim 1, wherein the syringe securing mechanism comprises a V-shaped clamp opposite the V-shaped notch and is configured to engage the syringe to secure the syringe in the V-shaped notch.

3. The syringe fixture of claim 2, wherein the syringe securing mechanism further comprises a syringe clamp removably coupled to the support block and connected to the V-shaped clamp.

4. The syringe fixture of claim 3, wherein the syringe securing mechanism further comprises a spring disposed between the syringe clamp and the V-shaped clamp, the spring biasing the V-shaped clamp away from the syringe clamp.

5. The syringe fixture of claim 1, wherein the support block comprises a support platform defining the V-shaped notch and a plurality of legs with proximal ends attached to the support platform and distal ends disposed away from the support platform, the distal ends having distal end surfaces collectively defining the first planar support surface.

6. The syringe fixture of claim 5, wherein at least one of the plurality of legs defines a side surface disposed perpendicular to the distal end surfaces, the side surface at least partly defining the second planar support surface.

7. The syringe fixture of claim 1, wherein the support block comprises a first color and the syringe securing mechanism comprises a second color distinct from the first color.

8. The syringe fixture of claim 1, wherein the first orientation is a vertical orientation and the second orientation is a horizontal orientation.

9. A system for measuring at least one geometric dimension of a syringe, the system comprising:
a syringe imaging system having a central imaging axis and a stage planar surface;
a syringe fixture including a support block and a first planar support surface, the syringe fixture configured to secure a syringe in a first orientation relative to the central imaging axis when the support block is positioned on the first planar support surface;
an image sensor configured to capture a first image of at least a portion of the syringe when secured by the syringe fixture and positioned on the first planar surface; and
one or more processors configured to generate at least one geometric measurement of the syringe based upon at least the first image.

10. The system of claim 9, wherein the syringe fixture comprises:
the support block defining a V-shaped notch, the first planar support surface, and a second planar support surface,
the first planar support surface having a first edge,
the second planar support surface having a second edge, wherein at least a portion of the second edge conjoins at least a portion of the first edge, and wherein the second planar support surface is perpendicular to the first planar support surface; and
a syringe securing mechanism coupled to the support block and configured to secure a syringe in a fixed position in the V-shaped notch such that the syringe occupies a first orientation when the support block is positioned on the first planar support surface and a second orientation perpendicular to the first orientation when the support block is positioned on the second planar support surface.

11. The system of claim 9, wherein the syringe fixture comprises a luer cap.

12. The system of claim 9, further comprising at least one of:
(a) a light probe configured to be inserted into a barrel of the syringe,
(b) a backlight disposed opposite the stage planar surface from the image sensor, or
(c) a display device configured to display the at least one geometric dimension of the syringe.

13. (canceled)

14. (canceled)

15. A method for measuring a geometric dimension of a syringe, the method comprising:
securing a syringe in a syringe fixture;
placing the syringe and syringe fixture in a first position on a stage planar surface of an imaging system, the syringe having a central syringe axis occupying a first predetermined orientation relative to a central image axis of the imaging system;
acquiring, with an image sensor of the imaging system, at least one image of at least a portion of the syringe; and
calculating, with a processor of the imaging system, at least a first geometric dimension of the syringe based on the at least one image.

16. The method of claim 15, wherein securing the syringe in a syringe fixture comprises securing a luer cap onto a distal end of the syringe and wherein placing the syringe and syringe fixture on the stage planar surface comprises placing the syringe in a vertical orientation supported by the luer cap on the stage planar surface.

17. The method of claim 15, wherein securing the syringe in a syringe fixture comprises securing the syringe in a V-shaped notch of a support block of the syringe fixture.

18. The method of claim 17, further comprising engaging the syringe with a V-shaped clamp opposite the V-shaped notch to secure the syringe in the V-shaped notch.

19. The method of claim 18, further comprising at least one of:
(a) removably coupling a syringe clamp to the support block, the syringe clamp connected to the V-shaped clamp, or
(b) biasing the V-shaped clamp away from the syringe clamp with a spring disposed between the syringe clamp and the V-shaped clamp.

20. (canceled)

21. The method of claim 17, wherein the support block further comprises a first planar support surface, the first planar surface in contact with the stage planar surface when the syringe and syringe fixture occupy the first position and the first predetermined orientation.

22. The method of claim 21, wherein the support block further comprises a second planar support surface that is perpendicular to the first planar support surface, the second planar surface in contact with the stage planar surface when the syringe and syringe fixture occupy a second position having a second predetermined orientation that is perpendicular to the first predetermined orientation.

23. The method of claim 22, wherein the support block comprises a support platform defining the V-shaped notch and a plurality of legs with proximal ends attached to the support platform and distal ends disposed away from the support platform, the distal ends having distal end surfaces collectively defining the first planar surface, and wherein at least one of the plurality of legs defines a side surface disposed perpendicular to the distal end surfaces, the side surface at least partly defining the second planar surface.

24. The method of claim 22, wherein the first predetermined orientation is a vertical orientation and the second predetermined orientation is a horizontal orientation.

25. The method of claim 15, wherein calculating the at least first geometric dimension comprises calculating (a) a syringe barrel inside diameter, (b) a syringe flange diameter, (c) a luer outside diameter, (d) a location of a syringe dose line, (e) a syringe barrel length, (f) a syringe flange thickness, and/or (g) a syringe barrel outside diameter.

26-41. (canceled)
